(19) United States
(12) Patent Application Publication
(10) Pub. No.: US 2007/0160623 A1
(43) Pub. Date: Jul. 12, 2007

(54) Innate immune system-directed vaccines

(76) Inventors: Ruslan M. Medzhitov, Branford, CT (US); Elizabeth Kopp, Fairfield, CT (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 11/709,993

(22) Filed: Feb. 23, 2007

Related U.S. Application Data
(63) Continuation of application No. 10/319,854, filed on Dec. 13, 2002, now abandoned.
Application No. 10/319,854 is a continuation-in-part of application No. 09/752,832, filed on Jan. 3, 2001, now abandoned.
(60) Provisional application No. 60/340,174, filed on Dec. 14, 2001.

(51) Int. Cl. A61K 39/00; C07H 21/04; C12P 21/04; C07K 14/47

(52) U.S. Cl. 424/185.1; 530/350; 536/23.5; 435/69.7; 435/320.1; 435/325

(57) ABSTRACT

The present invention provides novel vaccines, methods for the production of such vaccines and methods of using such vaccines. The novel vaccines of the present invention combine both of the signals necessary to activate native T-cells—a specific antigen and the co-stimulatory signal—leading to a robust and specific T-cell immune response.

### RELATED APPLICATIONS

This application is a continuation in part of U.S. application Ser. No. 09/752,832 (Pub. No.: U.S. 2002/0061312 A1), entitled Innate Immune System-Directed. Vaccines, by Rusian Medzhitov (filed Jan. 3, 2001) and claims the benefit of the filling date of U.S. Provisional Application No. 60/340,174 entitled fimC is a Novel Pamp and a Candidate for Recombinant Fusion Vaccines, by Rusian Medzhitov and Elizabeth Kopp (filed Dec. 14, 2001). The entire teachings of the referenced applications are expressly incorporated herein by reference.

### BACKGROUND OF THE INVENTION

All articles, patents and other materials referred to below are specifically incorporated herein by reference.

1. Immunity

Multicellular organisms have developed two general systems of immunity to infectious agents. The two systems are innate or natural immunity (also known as “innate imnmunity”) and adaptive (acquired) or specific immunity. The major difference between the two systems is the mechanism by which they recognize infectious agents.

The innate immune system uses a set of germline-encoded receptors for the recognition of conserved molecular patterns present in microorganisms. These molecular patterns occur in certain constituents of microorganisms including: lipopolysaccharides, peptidoglycans, lipoteichoic acids, phosphatidyl cholines, bacteria-specific proteins, including lipoproteins, bacterial DNAs, viral single and double-stranded RNAs, unmethylated CpG-DNAs, mannans and a variety of other bacterial and fungal cell wall components. Such molecular patterns can also occur in other molecules such as plant alkaloids. These targets of innate immune recognition are called Pathogen Associated Molecular Patterns (PAMPs) since they are produced by microorganisms and not by the infected host organism. (Janeway et al. (1989) Cold Spring Harb. Symp. Quant. Biol. 54: 1-13; Medzhitov et al. (1997) Curr. Opin. Immunol. 94: 4-9).

The receptors of the innate immune system that recognize PAMPs are called Pattern Recognition Receptors (PRRs). (Janeway et al. (1989) Cold Spring Harb. Symp. Quant. Biol. 54: 1-13; Medzhitov et al. (1997) Curr. Opin. Immunol. 94: 4-9). These receptors vary in structure and belong to several different protein families. Some of these receptors recognize PAMPs directly (e.g., CD14, DEC205, collectins), while others (e.g., complement receptors) recognize the products generated by PAMP recognition. Members of these receptor families can, generally, be divided into three types: 1) humoral receptors circulating in the plasma; 2) endocytic receptors expressed on immune-cell surfaces, and 3) signaling receptors that can be expressed either on the cell surface or intracellularly. (Medzhitov et al. (1997) Curr. Opin. Immunol. 94: 4-9; Fearon et al. (1996) Science 272: 50-3).

Cellular PRRs are expressed on effector cells of the innate immune system, including cells that function as professional antigen-presenting cells (APC) in adaptive immunity. Such effector cells include, but are not limited to, macrophages, dendritic cells, B lymphocytes and surface epithelia. This expression profile allows PRRs to directly induce innate effector mechanisms, and also to alert the host organism to the presence of infectious agents by inducing the expression of a set of endogenous signals, such as inflammatory cytokines and chemokines, as discussed below. This latter function allows efficient mobilization of effector forces to combat the invaders.

In contrast, the adaptive immune system, which is found only in vertebrates, uses two types of antigen receptors that are generated by somatic mechanisms during the development of each individual organism. The two types of antigen receptors are the T-cell receptor (TCR) and the immunoglobulin receptor (IgR), which are expressed on two specialized cell types, T-lymphocytes and B-lymphocytes, respectively. The specificities of these antigen receptors are generated at random during the maturation of lymphocytes by the processes of somatic gene rearrangement, random pairing of receptor subunits, and by a template-independent addition of nucleotides to the coding regions during the rearrangement.

Recent studies have demonstrated that the innate immune system plays a crucial role in the control of initiation of the adaptive immune response and in the induction of appropriate cell effector responses. (Fearon et al. (1996) Science 272: 50-3; Medzhitov et al (1997) Cell 91: 295-8). Indeed, it is now well established that the activation of naive T-lymphocytes requires two distinct signals: one is a specific antigenic peptide recognized by the TCR, and the other is the so called co-stimulatory signal, B7, which is expressed on APCs and recognized by the CD28 molecule expressed on T-cells. (Lenschow et al. (1996) Annu. Rev. Immunol. 14: 233-58). Activation of naive CD4⁺ T-lymphocytes requires that both signals, the specific antigen and the B7 molecule, are expressed on the same APC. If a naive CD4 T-cell recognizes the antigen in the absence of the B7 signal, the T-cell will die by apoptosis. Expression of B7 molecules on APCs, therefore, controls whether or not the naive CD4 T-lymphocytes will be activated. Since CD4 T-cells control the activation of CD8 T-cells for cytotoxic functions, and the activation of B-cells for antibody production, the expression of B7 molecules determines whether or not an adaptive immune response will be activated.

Recent studies have also demonstrated that the innate immune system plays a crucial role in the control of B7 expression. (Fearon et al. (1996) Science 272: 50-3; Medzhitov et al. (1997) Cell 91: 295-8). As mentioned earlier, innate immune recognition is mediated by PRRs that recognize PAMPs. Recognition of PAMPs by PRRs results in the activation of signaling pathways that control the expression of a variety of inducible immune response genes, including the genes that encode signals necessary for the activation of lymphocytes, such as B7, cytokines and chemokines. (Medzhitov et al. (1997) Cell 91: 295-8; Medzhitov et al. (1997) Nature 388: 394-397). Induction of B7 expression by PRR upon recognition of PAMPs thus accounts for self/nonself discrimination and ensures that only T-cells specific for microorganism-derived antigens are normally activated. This mechanism normally prevents activation of autoreactive lymphocytes specific for self-antigens.

Receptors of the innate immune system that control the expression of B7 molecules and cytokines have recently been identified. (Medzhitov et al. (1997) Nature 388: 394-397; Rock et al. (1998) Proc. Natl. Acad. Sci. USA, 95: 588-93). These receptors belong to the family of Toll-like receptors (TLRs), so called because they are homologous to the Drosophila Toll protein which is involved both in dorsoventral patterning in Drosophila embryos and in the immune response in adult flies. (Lemaitre et al. (1996) Cell 86: 973-83). In mammalian organisms, such TLRs have been shown to recognize PAMPs such as the bacterial products LPS, peptidoglycan, and lipoprotein. (Schwandner et al. (1999) J. Biol. Chem. 274: 17406-9; Yoshimura et al. (1999) J. Immunol. 163: 1-5; Aliprantis et al. (1999) Science 285: 736-9).

2. Vaccine Development

Vaccines have traditionally been used as a means to protect against disease caused by infectious agents. However, with the advancement of vaccine technology, vaccines have been used in additional applications that include, but are not limited to, control of mammalian fertility, modulation of hormone action, and prevention or treatment of tumors.

The primary purpose of vaccines used to protect against a disease is to induce immunological memory to a particular microorganism. More generally, vaccines are needed to induce an immune response to specific antigens, whether they belong to a microorganism or are expressed by tumor cells or other diseased or abnormal cells. Division and differentiation of B- and T-lymphocytes that have surface receptors specific for the antigen generate both specificity and memory.

In order for a vaccine to induce a protective immune response, it must fulfill the following requirements: 1) it must include the specific antigen(s) or fragment(s) thereof that will be the target of protective immunity following vaccination; 2) it must present such antigens in a form that can be recognized by the immune system, e.g., a form resistant to degradation prior to immune recognition; and 3) it must activate APCs to present the antigen to CD4+ T-cells, which in turn induce B-cell differentiation and other immune effector functions.

Conventional vaccines contain suspensions of attenuated or killed microorganisms, such as viruses or bacteria, incapable of inducing severe infection by themselves, but capable of counteracting the unmodified (or virulent) species when inoculated into a host. Usage of the term has now been extended to include essentially any preparation intended for active immunologic prophylaxis (e.g., preparations of killed microbes of virulent strains or living microbes of attenuated (variant or mutant) strains; microbial, fungal, plant, protozoan, or metazoan derivatives or products; synthetic vaccines). Examples of vaccines include, but are not limited to, cowpox virus for inoculating against smallpox, tetanus toxoid to prevent tetanus, whole-inactivated bacteria to prevent whooping cough (pertussis), polysaccharide subunits to prevent streptococcal pneumonia, and recombinant proteins to prevent hepatitis B.

Although attenuated vaccines are usually immunogenic, their use has been limited because their efficacy generally requires specific, detailed knowledge of the molecular determinants of virulence. Moreover, the use of attenuated pathogens in vaccines is associated with a variety of risk factors that in most cases prevent their safe use in humans.

The problem with synthetic vaccines, on the other hand, is that they are often non-immunogenic or non-protective. The use of available adjuvants to increase the immunogenicity of synthetic vaccines is often not an option because of unacceptable side effects induced by the adjuvants themselves.

An adjuvant is defined as any substance that increases the immunogenicity of admixed antigens. Although chemicals such as alum are often considered to be adjuvants, they are in effect akin to carriers and are likely to act by stabilizing antigens and/or promoting their interaction with antigen-presenting cells. The best adjuvants are those that mimic the ability of microorganisms to activate the innate immune system. Pure antigens do not induce an immune response because they fail to induce the costimulatory signal (e.g., B7.1 or B7.2) necessary for activation of lymphocytes. Thus, a key mechanism of adjuvant activity has been attributed to the induction of costimulatory signals by microbial, or microbial-like, constituents carrying PAMPs that are routine constituents of adjuvants. (Janeway et al. (1989) Cold Spring Harb. Symp. Quant. Biol., 54: 1-13). As discussed above, the recognition of these PAMPs by PRRs induces the signals necessary for lymphocyte activation (such as B7) and differentiation (effector cytokines).

Because adjuvants are often used in molar excess of antigens and thus trigger an innate immune response in many cells that do not come in contact with the target antigen, this non-specific induction of the innate immune system to produce the signals that are required for activation of an adaptive immune response produces an excessive inflammatory response that renders many of the most potent adjuvants clinically unsuitable. Alum is currently approved for use as a clinical adjuvant, even though it has relatively limited efficacy, because it is not an innate immune stimulant and thus does not cause excessive inflammation.

3. Alternative Vaccine Strategies

Immune Stimulating Complexes for Use as Vaccines. Immune stimulating complexes (ISCOMS) are cage-like structures comprising Quil-A, cholesterol, adjuvant active saponin and phospholipids that induce a wide range of systemic immune responses. (Mowat et al. (1999) Immunol. Lett. 65: 133-140; Smith et al., (1999) J. Immunol. 162(9): 5536-5546). ISCOMS are suitable for repeated administration of different antigens to an individual because these complexes allow the entry of antigen into both MHC I and II processing pathways. (Mowat et al. (1991) Immunol. 72: 317-322).

ISCOMS have been used with conjugates of modified soluble proteins. (Reid (1992) Vaccine 10(9): 597-602). These complexes also produce a Th1 type response, as would be expected for such a vaccine. (Morein et al. (1999) Methods 19: 94-102). Since ISCOMS do not specifically target APCs, their use can result in problems of toxicity and a lack of specificity.

Multiple Antigenic Recombinant Vaccines. Various U.S. patents disclose chimeric proteins consisting of multiple antigenic peptides (MAPs) for use as vaccines. For example, Klein et al. were granted a family of patents (e.g., U.S. Pat. Nos. 6,033,668; 6,017,539; 5,998,169; and 5,968,776) which describe genes encoding multimeric hybrids comprising an immunogenic region of a protein from a first antigen linked to an immunogenic region from a second pathogen. The vaccines contemplated by Klein et al. are fusion proteins, in which the component peptides are all selected by virtue of their being antigens (i.e., being recognized by a TCR or IgR). Thus the vaccines described by Klein et al. are not designed to stimulate the innate immune system.

Although many types of vaccines are available, it would be advantageous to have vaccines which provide greater protection.

### SUMMARY OF THE INVENTION

The novel vaccines of the present invention comprise one or more isolated PAMPs in combination with one or more antigens. The antigens used in the vaccines of the present invention can be any type of antigen (e.g., including but not limited to pathogen-related antigens, tumor-related antigens, allergy-related antigens, neural defect-related antigens, cardiovascular disease antigens, rheumatoid arthritis-related antigens, other disease-related antigens, hormones, pregnancy-related antigens, embryonic antigens and/or fetal antigens and the like). Examples of various types of vaccines, which can be produced by the present invention, are provided in FIG. 1.

In one embodiment, the vaccines are recombinant proteins, or recombinant lipoproteins, or recombinant glycoproteins, which contain a PAMP (e.g., BLP, Flagellin or FimC) and one or more antigens. The basic concept for preparing a fusion protein of the present invention is provided in FIG. 1.

Upon administration into human or animal subjects, the vaccines of the present invention will interact with APCs, such as dendritic cells and macrophages. This interaction will have two consequences: First, the PAMP portion of the vaccine will interact with a PRR such as a TLR and stimulate a signaling pathway, such as the NF-κB, JNK and/or p38 pathways. Second, due to the PAMP's interaction with TLRs and/or other pattern-recognition receptors, the recombinant vaccine will be taken up into dendritic cells and mactophages by phagocytosis, endocytosis, or macropinocytosis, depending on the cell type, the size of the recombinant vaccine, and the identity of the PAMP.

Activation of TLR-induced signaling pathways will lead to the induction of the expression of cytokines, chemokines, adhesion molecules, and co-stimulatory molecules by dendritic cells and macrophages and, in some cases, B-cells. Uptake of the vaccines will lead to the processing of the antigen(s) fused to the PAMP and their presentation by the MHC class-I and MHC class-II molecules. This will generate the two signals required for the activation of naive T-cells—a specific antigen signal and the co-stimulatory signal. In addition, chemokines induced by the vaccine (due to PAMP interaction with TLR) will recruit naive T-cells to the APC and cytokines, like IL-12, which will induce T-cell differentiation into Th-1 effector cells. As a result, a robust T-cell immune response will be induced, which will in turn activate other aspects of the adaptive immune responses, such as activation of antigen-specific B-cells and macrophages.

Thus, the novel vaccines of the present invention provide an efficient way to produce an immune response to one or more specific antigens without the adverse side effects normally associated with conventional vaccines.

The present invention relates generally to vaccines, methods of making vaccines and methods of using vaccines.

More specifically, the present invention provides vaccines comprising an isolated PAMP, immunostimulatory portion or immunostimulatory derivative thereof and an antigen or an immunogenic portion or immunogenic derivative thereof. An example of a vaccine of the present invention is a fusion protein comprising a PAMP, immunostimulatory portion or immunostimulatory derivative thereof and an antigen or an immunogenic portion or immunogenic derivative thereof.

The vaccines of the present invention can comprise any PAMP peptide or protein, including, but not limited to, the following PAMPs: peptidoglycans, lipoproteins and lipopeptides, Flagellins, chaperones, outer membrane proteins (OMPs), outer surface proteins (OSPs), other protein components of the bacterial cell walls, and other PRR ligands.

One PAMP of the present invention is BLP, including the BLP encoded by the polypeptide of SEQ ID NO: 2, set forth in FIG. 15. In addition to protein PAMPs, also useful in the vaccines of the present invention are peptide mimetics of any non-protein PAMP.

Antigens useful in the present invention include, but are not limited to, those that are microorganism-related, and other disease-related antigens, including but not limited to those that are allergen-related and cancer-related. The antigen component of the vaccine can be derived from sources that include, but are not limited to, bacteria, viruses, fungi, yeast, protozoa, metazoa, tumors, malignant cells, abnormal neural cells, arthritic lesions, cardiovascular lesions, plants, animals, humans, allergens, hormones and amyloid-β peptide. The antigens, immunogenic portions or immunogenic derivatives thereof can be composed of peptides, polypeptides, lipoproteins, glycoproteins, mucoproteins and the like.

The various vaccines of the present invention include, but are not limited to:

1) one or more PAMPs, immunostimulatory portions or immunostimulatory derivatives thereof, conjugated to one or mote antigens, immunogenic portions or immunogenic derivatives thereof;

2) a PAMP/antigen fusion protein comprising one or more PAMPs, immunostimulatory portions or immunostimulatory derivatives thereof, and one or more antigens, immunogenic portions or immunogenic derivatives thereof; and

3) a modified antigen, immunogenic portion or immunogenic derivative thereof that comprises a leader sequence fused to a lipidation or glycosylation consensus sequence that is further fused to the antigen, or an immunogenic portion or immunogenic derivative thereof.

The present invention also encompasses such vaccines further comprising a pharmaceutically acceptable carrier, including, but not limited to, alum.

More specifically, the present invention provides fusion proteins comprising one or more PAMPs, immunostimulatory portions or immunostimulatory derivatives thereof, and one or more antigens, immunogenic portions or immunogenic derivatives thereof. The PAMP domains of the fusion proteins of the present invention can be composed of amino acids, amino acid polymers, peptidoglycans, glycoproteins, and lipoproteins or any other suitable component. One preferred PAMP to use in the fusion proteins of the present invention is BLP, including the BLP encoded by the polypeptide of SEQ ID NO: 2. Flagellin is another PAMP to use in the fusion proteins of the present invention, and is provided by (but not limited to) accession numbers P04949 (E. Coli Flagellin) and A24262 (Salmonella Flagellin). Another PAMP to use in the fusion proteins of the present invention is FimC, and is provided by (but not limited to) the amino acid sequence of SEQ ID NO: 14 (accession number AAC77272) and the amino acid sequence encoded by the nucleic acid sequence of SEQ ID NO: 15 (accession number L14598). Useful antigen domain(s) in the fusion proteins of the present invention include, but are not limited to, Eα (a peptide antigen derived from mouse MHC class-II I-E), listeriolysin, PSMA, HIV gp120, Ra5G and TRP-2. In one embodiment, the fusion proteins of the present invention include a construct comprising the following components: a leader peptide that signals lipidation or glycosylation of the consensus sequence, a lipidation and/or glycosylation consensus sequence, and antigen. More specifically, the fusion proteins of the present invention include a construct comprising a leader sequence-CXXN-antigen, wherein the leader peptide is a signal for lipidation of the consensus sequence and wherein X is any amino acid, preferably serine. Examples of leader peptides useful in the present invention include, but are not limited to, those selected from the peptides of SEQ ID NO: 3 (shown in FIG. 15), SEQ ID NO: 4 (shown in FIG. 16), SEQ ID NO: 5 (shown in FIG. 17), SEQ ID NO: 6 (shown in FIG. 18) and SEQ ID NO: 7 (shown in FIG. 19).

In another embodiment, the present invention also provides a fusion protein comprising an isolated PAMP and an antigen, wherein the antigen is a self-antigen.

The present invention further provides methods of recombinantly producing the fusion proteins of the present invention. Thus, the present invention provides recombinant expression vectors comprising a nucleotide sequence encoding the chimeric constructs of the present invention as well as host cells transformed with such recombinant expression vectors. Any cell that is capable of expressing the fusion proteins of the present invention is suitable for use as a host cell. Such host cells include, but are not limited to, the cells of bacteria, yeast, insects, plants and animals. More preferably for certain PAMPs such as BLP, the host cell is a bacterial cell. Even more preferably, the host cell is a bacterial cell that can appropriately modify (e.g., lipidation, glycosylation) the PAMP domain of the fusion protein when such modification is necessary or desirable.

The present invention also provides methods of immunizing an animal with the vaccines of the present invention, where such methods include, but are not limited to, administering a vaccine parenterally, intravenously, orally, using suppositories, or via the mucosal surfaces. In one embodiment the animal being vaccinated is a human.

The immune response can be measured using any suitable method including, but not limited to, direct measurement of peripheral blood lymphocytes, natural killer cell cytotoxicity assays, cell proliferation assays, immunoassays of immune cells and subsets, and skin tests for cell-mediated immunity.

The present invention also provides methods of treating a patient susceptible to an allergic response to an allergen by administering a vaccine of the present invention and thereby stimulating the TLR-mediated signaling pathway.

The present invention also provides methods of treating a patient susceptible to or suffering from Alzheimer's disease by administering a vaccine of the present invention in which the target antigen is a peptide or protein associated with Alzheimer's disease, including but not limited to amyloid-β peptide.

The present invention further provides a method of stimulating an innate immune response in an animal and thereby enhancing the adaptive immune response to a foreign or self-antigen which comprises co-administering a PAMP with the foreign or self antigen.

The present invention also provides a vaccine which comprises a PAMP conjugated with a foreign or self antigen that stimulates an innate immune response in an animal and thereby enhances the adaptive immune response to a foreign or self-antigen but does not lead to undesirable levels of inflammation.

Additionally, the present invention provides a vaccine which comprises a PAMP conjugated with a foreign or self antigen which, when administered at a therapeutically active dose, stimulates an innate immune response in an animal and thereby enhances the adaptive immune response to a foreign or self-antigen but does not lead to undesirable levels of inflammation.

The present invention also provides a method of treatment comprising the steps of administering to an individual a vaccine which comprises a PAMP conjugated with a foreign or self antigen which stimulates an innate immune response in an animal and thereby enhances the adaptive immune response to a foreign or self-antigen but does not lead to undesirable levels of inflammation.

Additional embodiments of the present invention will be obvious to those skilled in the art of vaccine preparation and vaccine administration. Such obvious variations of the present invention are also contemplated by the present inventor.

### BRIEF DESCRIPTION OF THE DRAWINGS

FIG. 1 shows examples of alternative fusion proteins according to the present invention. Permutations and combinations of these fusion proteins can also be prepared according to the methods of the present invention.

FIG. 2 shows a basic outline for generating different recombinant protein vaccines containing different antigens and a signal to trigger the innate immune response (PAMP). Each antigen is represented by a different shape of the central portion of the vaccine.

FIG. 3 shows the BLP/Eα construct.

FIG. 4 shows that BLP/Eα activates NF-κB in dose-dependent manner.

FIG. 5 shows IL-6 production by dendritic cells stimulated with BLP/Eα.

FIG. 6 shows the induction of dendritic cell activation and vaccine antigen processing and presentation by the MHC class-II pathway.

FIG. 7 shows the immunostimulatory effect of the chimeric construct BLP/Eα on specific T-cells in vitro.

FIG. 8 shows the effect of the chimeric construct, BLP/Eα on specific T-cell proliferation in vivo.

FIG. 9 shows that CpG-induced B-cell activation is dependent upon MyD88. MyD88^{-/-}, MyD88-deficient cells; ICE^{-/-}, caspase-1-deficient cells; B10/ScCr, TLR4-deficient cells derived from C57BL/10ScCr mice; TLR2^{-/-}, TLR2-deficient cells.

FIG. 10 shows that IL-6 production by macrophages during CpG stimulation and CpG-DNA-induced IkBα degradation is mediated by a signaling pathway dependent on MyD88.

FIG. 11 shows that wild-type and B10/ScCr dendritic cells, but not dendritic cells from MyD88^{-/-} animals produce IL-12 when stimulated with CpG oligonucleotides.

FIG. 12 shows activation of NF-κB by Flagellin fusions.

FIG. 13 shows induction of NF-κB in macrophages by Flagellin fusions.

FIG. 14 shows NF-κB activity in RAW κB cells.

FIG. 15 shows SEQ ID NO: 3.

FIG. 16 shows SEQ ID NO: 4.

FIG. 17 shows SEQ ID NO: 5.

FIG. 18 shows SEQ ID NO: 6.

FIG. 19 shows SEQ ID NO: 7.

FIG. 20 shows SEQ ID NO: 10.

FIG. 21 shows SEQ ID NO: 11.

FIG. 22 shows activation of NF-κB by FimC.

FIG. 23 shows that boiling reduces the activity of FimC.

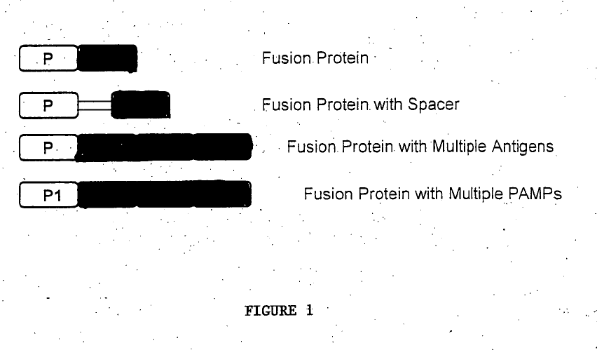

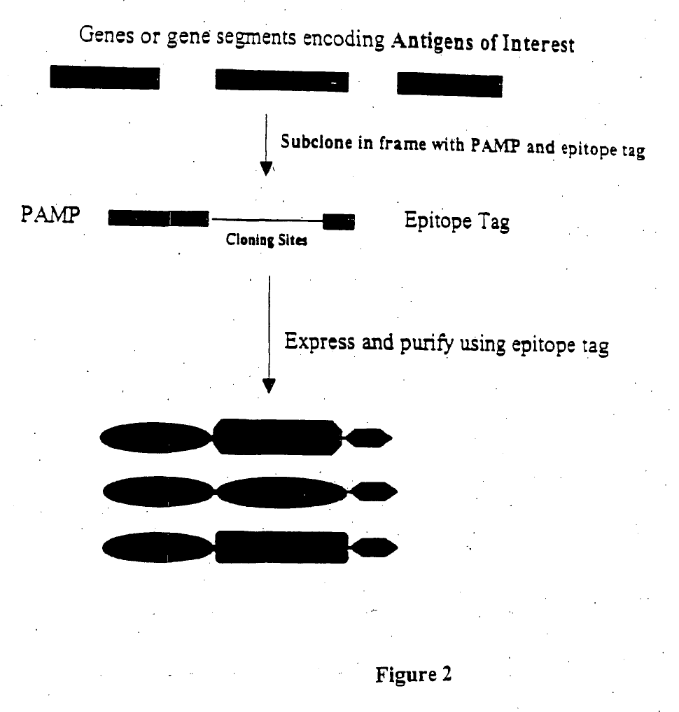

### DETAILED DESCRIPTION OF THE INVENTION

1. General Description

The present invention discloses a novel strategy of vaccine design based on the inventor's recent findings in the field of innate immunity. This approach is not limited to any particular antigen or immunogenic portions or derivatives thereof (e.g., microorganism-related, allergen-related or tumor-related, and the like) nor is it limited to any particular PAMP or immunostimulatory portions or immunostimulatory derivatives thereof. The term “vaccine”, therefore, is used herein in a general sense to refer to any therapeutic or immunogenic or immunostimulatory composition that includes the features of the present invention. A more detailed definition of vaccine is disclosed elsewhere herein.

The activation of an adaptive immune response requires both the specific antigen or derivative thereof, and a signal (e.g. PAMP) that can induce the expression of B7 on the APCs. The present invention combines, in a single chimeric construct, both signals required for the induction of the adaptive immune responses—a signal recognized by the innate immune system (PAMP), and a signal recognized by an antigen receptor (antigen).

Fusion of an antigen with a PAMP, such as bacterial lipoprotein (BLP), optimizes the stoichiometry of the two signals and coordinates their effect on the same APC, thus minimizing the unwanted excessive inflammatory responses that occur when antigens are mixed with adjuvants comprising innate immune stimulants to increase their immunogenicity. In addition, the chimeric constructs of the present invention will prevent activation of APCs that do not take up the antigen. Activation of such APCs in the absence of uptake and presentation of the target antigen can lead to the induction of autoimmune responses, which, again, is one of the problems with commonly used innate immunity-stimulating adjuvants that prevents or limits their use in humans. Notably, the chimeric constructs of the present invention exhibit the essential immunological characteristics or properties expected of a conventional vaccine supplemented with an adjuvant, but the chimeric constructs do not induce an excessive inflammatory reaction as is often induced by an adjuvant. Thus, the vaccine approach described in the present invention minimizes or eliminates undesired side effects (e.g., excessive inflammatory reaction, autoimmunity) yet induces a very potent and specific immune response, and provides a favorable alternative to vaccines comprising mixtures of antigens and adjuvants.

According to the present invention, neither the PAMP nor the antigen need consist of a polypeptide. However, either the PAMP or the antigen, or both, may be a peptide or polypeptide. In one embodiment of the present invention, recombinant DNA technology may be utilized in the production of chimeric constructs, for use in vaccines, when both the PAMP, or an immunogenic portion or derivative thereof, and the antigen, or an immunostimulatory portion or derivative thereof, are polypeptides. Alternatively, recombinant techniques may also be utilized to produce a protein chimeric construct when a peptide mimetic is used in lieu of a non-protein antigen, such as a polysaccharide or a nucleic acid and the like, and/or a non-protein PAMP, such as a lipopolysaccharide, CpG-DNA, bacterial DNA, single or double-stranded viral RNA, phosphatidyl choline, lipoteichoic acids and the like, for example. The present invention contemplates in one embodiment the use of BLP, the bacterial outer membrane proteins (OMP), the outer surface proteins A (OspA) of bacteria, Flagellins, chaperones including periplasmic chaperones such as FimC and other DNA-encoded PAMPs in the recombinant production of chimeric constructs. These PAMPs are known to induce activation of the innate immune response and therefore would be particularly suitable for use in vaccine formulations. (Henderson et al. (1996) Microbiol. Rev. 60: 31641). Furthermore, BLP has been shown to be recognized by TLRs. (Aliprantis et al. (1999) Science 285: 736-9). The details of the approach are described using BLP as the PAMP domain of a PAMP/antigen fusion protein; however any inducers of the innate immune system are equally applicable for such purpose in the present invention.

In another embodiment, one or more PAMP mimetics is substituted in place of a PAMP in a fusion protein.

This invention further provides methods for producing chimeric constructs where either the PAMP or an immunostimulatory portion or derivative thereof, or the antigen or an immunogenic portion or derivative thereof, or both the PAMP and the antigen are non-protein. Generally, these methods utilize chemical means to conjugate a PAMP to an antigen thereby producing a non-protein chimeric construct.

This invention further provides ways to exploit recombinant DNA technology in the synthesis of the peptide vaccines. Many of the surface antigens present on the pathogens of interest would not be amenable to encoding by nucleic acids as they are not proteins (e.g., lipopolysaccharides) or possess low protein content (e.g., peptidoglycans).

The present invention contemplates the use of peptide mimetics for these surface antigens or an immunogenic protein or derivative thereof, and the use of peptide mimetics in vaccines.

As discussed in greater detail herein, the present invention contemplates vaccines comprising chimeric constructs that comprise at least one antigen, or an immunogenic portion or derivative thereof, and at least one PAMP, or an immunogenic portion or derivative thereof. Thus, the present invention encompasses vaccines comprising fusion proteins where one or more protein antigens are linked to one or more protein PAMPs or a peptide mimetic of a PAMP. Preferably, the fusion protein has maximal immunogenicity and induces only a modest inflammatory response.

In instances in which a target antigen, or a domain of a target antigen, has a relatively low molecular weight and is not adequately immunogenic because of its small size, that antigen or antigen domain can act as a hapten and can be combined with a larger carrier molecule such that the molecular weight of the combined molecule will be high enough to evoke a strong immune response against the antigen. In one embodiment of this invention, the antigen itself serves as the carrier molecule. In another embodiment of this invention, the PAMP serves as the carrier molecule. In yet another embodiment, a hapten is combined, by either fusion or conjugation, with the PAMP or the antigen domain of the vaccine to elicit an antibody response to the hapten. In yet another embodiment, which would, without limitation, be preferable when the molecular weight of both antigen and PAMP are low, the PAMP and the antigen are combined with a third molecule that serves as the carrier molecule. Such carrier molecule can be keyhole limpet hemocyanin or any of a number of carrier molecules for haptens that are known to the artisan. In yet another embodiment, a fusion protein contains an antigen or antigen domain, a PAMP or a portion of a PAMP or a PAMP mimetic, and a carrier protein or carrier peptide. Once again, such carrier protein can be keyhole limpet hemocyanin or any of a number of carrier proteins or carrier peptides for haptens that are known to the artisan. Increasing the number of antigens or antigen epitopes, by using multiple antigen proteins and/or multiple domains of the same antigen protein or of different antigen proteins and/or some combination of the foregoing, are contemplated in this invention. Also contemplated are fusion proteins in which the number of PAMPs or PAMP derivatives or PAMP mimetics is increased. It is within the skill of the artisan to determine the optimal ratio of PAMP to antigen domains to maximize immunogenicity and minimize inflammatory response.

2. Definitions

“Adaptive immunity” refers to the adaptive immune system, which involves two types of receptors generated by somatic mechanisms during the development of each individual organism. As used herein, the “adaptive immune system” refers to both cellular and humoral immunity. Immune recognition by the adaptive immune system is mediated by antigen receptors.

“Adaptive immune response” refers to a response involving the characteristics of the “adaptive immune system” described above.

“Adapter molecule” refers to a molecule that can be transiently associated with some TLRs, mediates immunostimulation by molecules of the innate immune system, and mediates cytokine-induced signaling. “Adapter molecule” includes, but is not limited to, myeloid differentiation marker 88 (MyD88).

“Allergen” refers to an antigen, or a portion or derivative of an antigen, that induces an allergic or hypersensitive response.

“Amino acid polymer” refers to proteins, or peptides, and other polymers comprising at least two amino acids linked by a peptide bond(s), wherein such polymers contain either no non-peptide bonds or one or more non-peptide bonds. As used herein, “proteins” include polypeptides and oligopeptides.

“Antigen” refers to a substance that is specifically recognized by the antigen receptors of the adaptive immune system. Thus, as used herein, the term “antigen” includes antigens, derivatives or portions of antigens that are immunogenic and immunogenic molecules derived from antigens. Preferably, the antigens used in the present invention are isolated antigens. Antigens that are particularly useful in the present invention include, but are not limited to, those that are pathogen-related, allergen-related, or disease-related.

“Antigenic determinant” refers to a region on an antigen at which a given antigen receptor binds.

“Antigen-presenting cell” or “APC” or “professional antigen-presenting cell” or “professional APC” is a cell of the immune system that functions in triggering an adaptive immune response by taking up, processing and expressing antigens on its surface. Such effector cells include, but are not limited to, macrophages, dendritic cells and B cells.

“Antigen receptors” refers to the two types of antigen receptors of the adaptive immune system: the T-cell receptor (TCR) and the immunoglobulin receptor (IgR), which are expressed on two specialized cell types, T-lymphocytes and B-lymphocytes, respectively. The secreted form of the immunoglobulin receptor is referred to as antibody. The specificities of the antigen receptors are generated at random during the maturation of the lymphocytes by the processes of somatic gene rearrangement, random pairing of receptor subunits, and by a template-independent addition of nucleotides to the coding regions during the rearrangement.

“Chimeric construct” refers to a construct comprising an antigen and a PAMP, or PAMP mimetic, wherein the antigen and the PAMP are comprised of molecules such as amino acids, amino acid polymers, nucleotides, nucleotide polymers, carbohydrates, carbohydrate polymers, lipids, lipid polymers or other synthetic or naturally occurring chemicals or chemical polymers. As used herein, a “chimeric construct” refers to constructs wherein the antigen is comprised of one type of molecule in association with a PAMP or PAMP mimetic, which is comprised of either the same type of molecule or a different type of molecule.

“CpG” refers to a dinucleotide in which an unmethylated cytosine (C) residue occurs immediately 5′ to a guanosine (G) residue. As used herein, “CpG-DNA” refers to a synthetic CpG repeat, intact bacterial DNA containing CpG motifs, or a CpG-containing derivative thereof. The immunostimulatory effect of CpG-DNA on B-cells is mediated through a TLR and is dependent upon a “protein adapter molecule”.

“Derivative” refers to any molecule or compound that is structurally related to the molecule or compound from which it is derived. As used herein, “derivative” includes peptide mimetics (e.g., PAMP mimetics).

“Domain” refers to a portion of a protein with its own function. The combination of domains in a single protein determines its overall function. An “antigen domain” comprises an antigen or an immunogenic portion or derivative of an antigen. A “PAMP domain” comprises a PAMP or a PAMP mimetic or an immunostimulatory portion or derivative of a PAMP or a PAMP mimetic.

“Fusion protein” and “chimeric protein” both refer to any protein fusion comprising two or more domains selected from the following group consisting of: proteins, peptides, lipoproteins, lipopeptides, glycoproteins, glycopeptides, mucoproteins, mucopeptides, such that at least two of the domains are either from different species or encoded by different genes or such that one of the two domains is found in nature and the second domain is not known to be found in nature or such that one of the two domains resembles a molecule found in nature and the other does not resemble that same molecule. The term “fusion protein” also refers to an antigen or an immunogenic portion or derivative thereof which has been modified to contain an amino acid sequence that results in post-translational modification of that amino acid sequence or a portion of that sequence, wherein the post-translationally modified sequence is a ligand for a PRR. As yet another definition of a fusion protein, in the foregoing sentence, the amino acid sequence that results in post-translational modification to form a ligand for a PRR can comprise a consensus sequence, or that amino acid sequence can contain a leader sequence and a consensus sequence.

“Hapten” refers to a small molecule that is not by itself immunogenic but can bind antigen receptors and can combine with a larger carrier molecule to become immunogenic.

“In association with” refers to a reversible union between two chemical entities, whether alike or different, to form a more complex substance.

“In combination with” refers to either a reversible or irreversible (e.g. covalent) union between two chemical entities, whether alike or different, to form a more complex substance.

“Immunostimulatory” refers to the ability of a molecule to activate either the adaptive immune system or the innate immune system. As used herein, “antigens” are examples of molecules that are capable of stimulating the adaptive immune system, whereas PAMPs or PAMP mimetics are examples of molecules that are capable of stimulating the innate immune system. As used herein, “activation” of either immune system includes the production of constituents of humoral and/or cellular immune responses that are reactive against the immunostimulatory molecule.

“Innate immunity” refers to the innate immune system, which, unlike the “adaptive immune system”, uses a set of germline-encoded receptors for the recognition of conserved molecular patterns present in microorganisms.

“Innate immune response” refers to a response involving the characteristics of the “innate immune system” described above.

“Isolated” refers to a substance, cell, tissue, or subcellular component that is separated from or substantially purified with respect to a mixture or naturally occurring material.

“Linker” refers to any chemical entity that links one chemical moiety to another chemical moiety. Thus, something that chemically or physically connects a PAMP and an antigen is a linker. Examples of linkers include, but are not limited to, complex or simple hydrocarbons, nucleosides, nucleotides, nucleotide phosphates, oligonucleotides, polynucleotides, nucleic acids, amino acids, small peptides, polypeptides, carbohydrates (e.g., monosaccharides, disaccharides, trisaccharides), and lipids. Additional examples of linkers are provided in the Detailed Description Selection included herein. Without limitation, the present invention also contemplates using a peptide bond or an amino acid or a peptide linker to link a polypeptide PAMP and a polypeptide antigen. The present invention further contemplates preparing such a linked molecule by recombinant DNA procedures. A linker can also function as a spacer.

“Malignant” refers to an invasive, spreading tumor.

“Microorganism” refers to a living organism too small to be seen with the naked eye. Microorganisms include, but are not limited to bacteria, fungi, protozoans, microscopic algae, and also viruses.

“Mimetic” refers to a molecule that closely resembles a second molecule and has a similar effect or function as that of the second molecule.

“Moiety” refers to one of the component parts of a molecule. While there are normally two moieties in a single molecule, there may be more than two moieties in a single molecule.

“Molecular pattern” refers to a chemical structure or motif that is typically a component of microorganisms, or certain other organisms, but which is not typically produced by normal human cells or by other normal animal cells. Molecular patterns are found in, or composed of, the following types of molecules: lipopolysaccharides, peptidoglycans, lipoteichoic acids, phosphatidyl cholines, lipoproteins, bacterial DNAs, viral single and double-stranded RNAs, certain viral glycoproteins, unmethylated CpG-DNAs, mannans, and a variety of other bacterial, fungal and yeast cell wall components and the like.

“Non-protein chimeric construct” or “non-protein chimera” refers to a “chimeric construct” wherein either the antigen or the PAMP or the PAMP mimetic, or two or more of them, is not an amino acid polymer.

“Pathogen-Associated Molecular Pattem” or “PAMP” refers to a molecular pattern found in a microorganism but not in humans, which, when it binds a PRR, can trigger an innate immune response. Thus, as used herein, the term “PAMP” includes any such microbial molecular pattern and is not limited to those associated with pathogenic microorganisms or microbes. As used herein, the term “PAMP” includes a PAMP, derivative or portion of a PAMP that is immunostimulatory, and any immunostimulatory molecule derived from any PAMP. These structures, or derivatives thereof, are potential initiators of innate immune responses, and therefore, ligands for PRRs, including Toll receptors and TLRs. “PAMPs” are immunostimulatory structures that are found in, or composed of molecules including, but not limited to, lipopolysaccharides; phosphatidyl choline; glycans, including peptidoglycans; teichoic acids, including lipoteichoic acids; proteins, including lipoproteins and lipopeptides; outer membrane proteins (OMPs), outer surface proteins (OSPs) and other protein components of the bacterial cell walls and Flagellins; chaperones including periplasmic chaperones such as FimC; bacterial DNAs; single and double-stranded viral RNAs; unmethylated CpG-DNAs; mannans; mycobacterial membranes; porins; and a variety of other bacterial and fungal cell wall components, including those found in yeast. Additional examples of PAMPs are provided in the Detailed Description section included herein.

“PAMP/antigen conjugate” refers to an antigen and a PAMP or PAMP mimetic that are covalently or noncovalently linked. A conjugate may be comprised of a protein PAMP or antigen and a non-protein PAMP or antigen.

“PAMP/antigen fusion” or “PAMP/antigen chimera” refers to any protein fusion formed between a PAMP or PAMP mimetic and an antigen.

“Passive immunization” refers to the administration of antibodies or primed lymphocytes to an individual in order to confer immunity.

“PAMP mimetic” refers to a molecule that, although it does not occur in microorganisms, is analogous to a PAMP in that it can bind to a PRR and such binding can trigger an innate immune response. A PAMP mimetic can be a naturally-occurring molecule or a partially or totally synthetic molecule. As an example, and not by way of limitation, certain plant alkaloids, such as taxol, are PRR ligands, have an immunostimulatory effect on the innate immune system, and thus behave as PAMP mimetics. (Kawasaki et al. (2000) J. Biol. Chem. 275(4): 2251-2254).

“Pattern Recognition Receptor” or “PRR” refers to a member of a family of receptors of the innate immune system that, upon binding a PAMP, an immunostimulatory portion or derivative thereof, can initiate an innate immune response. Members of this receptor family are structurally different and belong to several different protein families. Some of these receptors recognize PAMPs directly (e.g., CD14, DEC205, collectins), while others (e.g., complement receptors) recognize the products generated by PAMP recognition. Members of these receptor families can, generally, be divided into three types: 1) humoral receptors circulating in the plasma; 2) endocytic receptors expressed on immune-cell surfaces, and 3) signaling receptors that can be expressed either on the cell surface or intracellularly. Cellular PRRs may be expressed on effector cells of the innate immune system, including cells that function as professional APCs in adaptive immunity, and also on cells such as surface epithelia that are the first to encounter pathogens during infection. PRRs may also induce the expression of a set of endogenous signals, such as inflammatory cytokines and chemokines. Examples of PRRs useful for the present invention include, but are not limited to, the following: C-type lectins (e.g., humoral, such as collectins (MBL), and cellular, such as macrophage C-type lectins, macrophage mannose receptors, DEC205); proteins containing leucine-rich repeats (e.g., Toll receptor and TLRs, CD14, RP105); scavenger receptors (e.g., macrophage scavenger receptors, MARCO, WCI); and pentraxins (e.g., C-reactive proteins, serum, amyloid P, LBP, BPIP, CD11b,C and CD18.

“Peptide mimetic” refers to a protein or peptide that closely resembles a non-protein molecule and has a similar effect or function to the non-protein molecule. Alternatively, a peptide mimetic can be a non-protein molecule or non-peptide molecule that closely resembles a peptide or protein and has a similar effect or function to the peptide or protein.

“Pharmaceutically acceptable carrier” refers to a carrier that can be tolerated by a recipient animal, typically a mammal.

“Protein chimeric construct” refers to a chimeric construct wherein both the antigen and the PAMP or PAMP mimetic are amino acid polymers.

“Recombinant” refers to genetic material that is produced by splicing genes, gene derivatives or other genetic material. As used herein, “recombinant” also refers to the products produced from recombinant genes (e.g. recombinant protein).

“Spacer” refers to any chemical entity placed between two chemical moieties that serves to physically separate the latter two moieties. Thus, a chemical entity placed between a PAMP or PAMP mimetic and an antigen is a spacer. Examples of spacers include, but are not limited to, nucleic acids (e.g. untranscribed DNA between two stretches of transcribed DNA), amino acids, carbohydrates (e.g., monosaccharides, disaccharides, trisaccharides), and lipids.

“Strong immune response” refers to an immune response, induced by the chimeric construct, that has about the same intensity or greater than the response induced by an antigen mixed with Complete Freund's Adjuvant (CFA).

“Therapeutically effective amount” refers to an amount of an agent (e.g., a vaccine) that can produce a measurable positive effect in a patient.

“Toll-like receptor” (TLR) refers to any of a family of receptor proteins that are homologous to the Drosophila melanogaster Toll protein. TLRs also refer to type I transmembrane signaling receptor proteins that are characterized by an extracellular leucine-rich repeat domain and an intracellular domain homologous to that of the interleukin 1 receptor. The TLR family includes, but is not limited to, mouse TLR2 and TLR4 and their homologues, particularly in other species including humans. This invention also defines Toll receptor proteins and TLRs wherein the nucleic acids encoding such proteins have at least about 70% sequence identity, more preferably, at least about 80% sequence identity, even more preferably, at least about 85% sequence identity, yet more preferably at least about 90% sequence identity, and most preferably at least about 95% sequence identity to the nucleic acid sequence encoding the Toll protein and the TLR proteins TLR2, TLR4 and other members of the TLR family. In addition, this invention also contemplates Toll receptors and TLRs wherein the amino acid sequences of such Toll receptors and TLRs have at least about 70% sequence identity, more preferably, at least about 80% sequence identity, even more preferably, at least about 85% sequence identity, yet more preferably at least about 90% sequence identity, and most preferably at least about 95% sequence identity to the amino acid sequences of the Toll protein and the TLRs, TLR2, TLR4 and their homologues.

“Tumor” refers to a mass of proliferating cells lacking, to varying degrees, normal growth control. As used herein, “tumors” include, at one extreme, slowly proliferating “benign” tumors, to, at the other extreme, rapidly proliferating “malignant” tumors that aggressively invade neighboring tissues.

“Vaccine” refers to a composition comprising an antigen, and optionally other ancillary molecules, the purpose of which is to administer such compositions to a subject to stimulate an immune response specifically against the antigen and preferably to engender immunological memory that leads to mounting of an immune response should the subject encounter that antigen at some future time. Examples of other ancillary molecules are adjuvants, which are non-specific immunostimulatory molecules, and other molecules that improve the pharmacokinetic and/or pharmacodynamic properties of the antigen. Conventionally, a vaccine usually consists of the organism that causes a disease (suitably attenuated or killed) or some part of the pathogenic organism as the antigen. Attenuated organisms, such as attenuated viruses or attenuated bacteria, are manipulated so that they lose some or all of their ability to grow in their natural host. There are now a range of biotechnological approaches used to producing vaccines. (See, e.g., W. Bains (1998) Biotechnology From A to Z, Second Edition, Oxford University Press). The various methods include, but are not limited to, the following:
1) Viral vaccines consisting of genetically altered viruses. The viruses can be engineered so that they are harmless but can still replicate (albeit inefficiently, sometimes) in cultured animal cells. Another approach is to clone the gene for a protein from a pathogenic virus into another, harmless virus, so that that resulting, engineered virus has certain immunologic properties of the pathogenic virus but does not cause any disease. Examples of the latter method include, but are not limited to, altered vaccinia and adenoviruses used as rabies vaccines for distribution with meat bait, and a vaccinia virus engineered to produce haemagglutinin and fusion proteins of rindepest virus of cattle;
2) Enhanced bacterial vaccines consisting of bacteria genetically engineered to enhance their value as vaccines when the bacteria are dead (e.g., E. coli vaccine for pigs, bacterial vaccine for furunculosis in salmon). Recombinant DNA techniques can be used to delete pathogenesis-causing genes in the bacteria or to engineer the protective epitope from a pathogen into a safe bacterium;
3) Biopharmaceutical vaccines consist of proteins, or portions of proteins, that are the same as the proteins in a viral, fungal or bacterial coat or wall; which can be made by recombinant DNA methods;
4) Multiple antigen peptides (MAPs) are peptide vaccines that are chemically attached (usually on a polylysine backbone), enabling several vaccines to be delivered at the same time;
5) Polyprotein vaccines consist of a single protein made by genetic engineering so that the different peptides from the organisms of interest form part of a continuous polypeptide chain; and
6) Vaccines produced in transgenic plants that can be used as food to provide oral vaccines (e.g., vaccine delivery by eating bananas).

3. Specific Embodiments

A. Fusion Proteins

The present invention is based in part on the unexpected discovery that vaccines comprising chimeric constructs of a PAMP and an antigen (e.g., the fusion protein BLP/Eα) exhibit the essential immunological characteristics or properties expected of a conventional vaccine supplemented with an adjuvant.

In one aspect, the present invention is based on the finding that BLP/Eα induces activation of NF-κB and production of IL-6 in macrophages and dendritic cells, respectively, demonstrating that the vaccine is capable of activating the innate immune system. The activity of BLP/Eα is comparable to that of LPS, and is not due to endotoxin contamination, as demonstrated by the lack of inhibition by polymyxin B.

In another aspect, the present invention is based on the finding that the BLP/Eα fusion protein induces maturation of dendritic cells, as demonstrated by the induction of the cell surface expression of the co-stimulatory molecule, B7.2. Additionally, BLP/Eα is appropriately targeted to the antigen processing and presentation pathway, and a functional Eα peptide/MHC class-II complex is generated. This result is demonstrated by FACS analysis using an antibody specific for the Eα peptide complexed with MHC class-II.

Moreover, the present invention is based on the surprising discovery that a recombinant vaccine comprising a BLP/Eα chimeric construct activates antigen-specific T-cell responses in vitro by stimulating dendritic cell activation and generating a specific ligand (Eα/MHC-II) for the T-cell receptor. Furthermore, the results of immunization of mice with BLP/Eα and the resultant antigen-specific T-cell responses demonstrate that the recombinant vaccine activates antigen-specific T-cell responses in vivo. For comparison, mice were immunized with Eα peptide mixed with Complete Freund's Adjuvant (CFA). The recombinant vaccine of the present invention induced an immune response in the mice that is stronger than that produced by Eα peptide mixed with CFA.

The present invention is also based on the surprising discovery that immunization with the recombinant vaccines that comprise the chimeric constructs of the present invention induce a minimal inflammatory reaction when compared to that induced by an adjuvant. However, as noted above, in spite of a reduced inflammatory response, the vaccine unexpectedly induced a strong immune response. Thus, the vaccine approach described in the present invention minimizes an undesired side effect (e.g., an excessive inflammatory reaction) yet induces a very potent and specific immune response. The present invention also provides fusion proteins comprising at least one antigen molecule or antigen domain and at least one PAMP or PAMP mimetic for use as vaccines. Preferably, the fusion protein has maximal immunogenicity and induces only a modest inflammatory response. Increasing the number of antigens or antigen epitopes, by using multiple antigen proteins and/or multiple domains of the same antigen protein or of different antigen proteins, and/or some combination of the foregoing, are contemplated in this invention. It is within the skill of the artisan to determine the optimal ratio of PAMP to antigen molecules to maximize immunogenicity and minimize or control the inflammatory response.

There are several advantages of using a fusion system for the production of recombinant polypeptides. First, heterologous proteins and peptides are often degraded by host proteases; this may be avoided, especially for small peptides, by using a gene fusion expression system. Second, general and efficient purification schemes are established for several fusion partners. The use of a fusion partner as an affinity handle allows rapid isolation of the recombinant peptide. Third, by using different fusion partners, the recombinant product may be localized to different compartments or it might be secreted; such strategy could lead to facilitation of purification of the fusion partner and/or directed compartmentalization of the fusion protein.

Additionally, various methods are available for chemical or enzymatic cleavage of the fusion protein that provides efficient strategies to obtain the desired cleavage product in large quantities. Frequently employed fusion systems are the Staphylococcal protein A fusion system and the synthetic ZZ variant which have IgG affinity and have been used for the generation of antibodies against short peptides; the glutathione S-transferase fusion system (Smith et al. (1988) Gene 60); the β-galactosidase fusion system; and the trpE fusion system (Yansura (1990) Methods Enzym. 185: 61). Some of these systems are commercially available as kits, including vectors, purification components and detailed instructions.

The present invention also contemplates modified fusion proteins having affinity for metal (metal ion) affinity matrices, whereby one or more specific metal-binding or metal-chelating amino acid residues are introduced, by addition, deletion, or substitution, into the fusion protein sequence as a tag. Optimally, the fusion partner, e.g., the antigen or PAMP sequence, is modified to contain the metal-chelating amino acid tag; however the antigen or PAMP could also be altered to provide a metal-binding site if such modifications could be achieved without adversely effecting a ligand-binding site, an active site, or other functional sites, and/or destroying important tertiary structural relationships in the protein. These metal-binding or metal-chelating residues may be identical or different, and can be selected from the group consisting of cysteine, histidine, aspartate, tyrosine, tryptophan, lysine, and glutamate, and are located so to permit binding or chelation of the expressed fusion protein to a metal. Histidine is the preferred metal-binding residue. The metal-binding/chelating residues are situated with reference to the overall tertiary structure of the fusion protein to maximize binding/chelation to the metal and to minimize interference with the expression of the fusion protein or with the protein's biological activity.

A fusion sequence of an antigen, PAMP and a tag may optionally contain a linker peptide. The linker peptide might separate a tag from the antigen sequence or the PAMP sequence. If the linker peptide so used encodes a sequence that is selectively cleavable or digestible by conventional chemical or enzymatic methods, then the tag can be separated from the rest of the fusion protein after purification. For example, the selected cleavage site within the tag may be an enzymatic cleavage site. Examples of suitable enzymatic cleavage sites include sites for cleavage by a proteolytic enzyme, such as enterokinase, Factor Xa, trypsin, collagenase, and thrombin. Alternatively, the cleavage site in the linker may be a site capable of cleavage upon exposure to a selected chemical (e.g., cyanogen bromide, hydroxylamine, or low pH).

Cleavage at the selected cleavage site enables separation of the tag from the antigen/PAMP fusion protein. The antigen/PAMP fusion protein may then be obtained in purified form, free from any peptide fragment to which it was previously linked for ease of expression or purification. The cleavage site, if inserted into a linker useful in the fusion sequences of this invention, does not limit this invention.

Any desired cleavage site, of which many are known in the art, may be used for this purpose.

The optional linker peptide in a fusion protein of the present invention might serve a purpose other than the provision of a cleavage site. As an example, and not by limitation, the linker peptide might be inserted between the PAMP and the antigen to prevent or alleviate steric hindrance between the two domains. In addition, the linker sequence might provide for post-translational modification including, but not limited to, e.g., phosphorylation sites, biotinylation sites, sulfation sites, carboxylation sites, lipidation sites, glycosylation sites and the like.

In one embodiment, the fusion protein of this invention contains an antigen sequence fused directly at its amino or carboxyl terminal end to the sequence of a PAMP. In another embodiment, the fusion protein of this invention, comprising an antigen and a PAMP sequence, is fused directly at its amino or carboxyl terminal end to the sequence of a tag. The resulting fusion protein is a soluble cytoplasmic fusion protein. In another embodiment, the fusion sequence further comprises a linker sequence interposed between the antigen sequence and a PAMP sequence or sequence of a tag. This fusion protein is also produced as a soluble cytoplasmic protein.

B. Antigens

As used herein, an “antigen” is any substance that induces a state of sensitivity and/or immune responsiveness after any latent period (normally, days to weeks in humans) and that reacts in a demonstrable way with antibodies and/or immune cells of the sensitized subject in vivo or in vitro. Examples of antigens include, but are not limited to, (1) microbial-related antigens, especially antigens of pathogens such as viruses, fungi or bacteria, or immunogenic molecules derived from them; (2) “self” antigens, collectively comprising cellular antigens including cells containing normal transplantation antigens and/or tumor-related antigens, RR-Rh antigens and antigens characteristic of, or specific to particular cells or tissues or body fluids; (3) allergen-related antigens such as those associated with environmental allergens (e.g., grasses, pollens, molds, dust, insects and dander), occupational allergens (e.g., latex, dander, urethanes, epoxy resins), food (e.g., shellfish, peanuts, eggs, milk products), drugs (e.g., antibiotics, anesthetics) and (4) vaccines (e.g., flu vaccine).

Antigen processing and recognition of displayed peptides by T-lymphocytes depends in large part on the amino acid sequence of the antigen rather than the three-dimensional structure of the antigen. Thus, the antigen portion used in the vaccines of the present invention can contain epitopes or specific domains of interest rather than the entire sequence. In fact, the antigenic portions of the vaccines of the present invention can comprise one or more immunogenic portions or derivatives of the antigen rather than the entire antigen. Additionally, the vaccine of the present invention can contain an entire antigen with intact three-dimensional structure or a portion of the antigen that maintains a three-dimensional structure of an antigenic determinant, in order to produce an antibody response by B-lymphocytes against a spatial epitope of the antigen.

1. Pathogen-Related Antigens. Specific examples of pathogen-related antigens include, but are not limited to, antigens selected from the group consisting of vaccinia, avipox virus, turkey influenza virus, bovine leukemia virus, feline leukemia virus, avian influenza, chicken pneumovirosis virus, canine parvovirus, equine influenza, FHV, Newcastle Disease Virus (NDV), Chicken/Pennsylvania/1/83 influenza virus, infectious bronchitis virus; Dengue virus, measles virus, Rubella virus, pseudorabies, Epstein-Barr Virus, HIV, SIV, EHV, BHV, HCMV, Hantaan, C. tetani, mumps, Morbillivirus, Herpes Simplex Virus type 1, Herpes Simplex Virus type 2, Human cytomegalovirus, Hepatitis A Virus, Hepatitis B Virus, Hepatitis C Virus, Hepatitis E Virus, Respiratory Syncytial Virus, Human Papilloma Virus, Influenza Virus, Salmonella, Neisseria, Borrelia, Chlamydia, Bordetella, and Plasmodium and Toxoplasma, Cryptococcus, Streptococcus, Staphylococcus, Haemophilus, Diptheria, Tetanus, Pertussis, Escherichia, Candida, Aspergillus, Entamoeba, Giardia, and Trypanasoma.

2. Cancer-Related Antigens. The methods and compositions of the present invention can also be used to produce vaccines directed against tumor-associated protein antigens such as melanoma-associated antigens, mammary cancer-associated antigens, colorectal cancer-associated antigens, prostate cancer-associated antigens and the like.

Specific examples of tumor-related or tissue-specific protein antigens useful in such vaccines include, but are not limited to, antigens selected from the group in the following table.

| Cancer type | Antigens |
|---|---|
| Prostate | prostate-specific antigen (PSA), |
| | prostate-specific membrane |
| | antigen (PSMA), Her-2neu, SPAS-1 |
| Melanoma | TRP-2, tyrosinase, Melan A/Mart-1, |
| | gp100, BAGE, GAGE, GM2 ganglioside |
| Breast | Her2-neu, kinesin 2, TATA element |
| | modulatory factor 1, tumor protein |
| | D52, MAGE D, ING2, HIP-55, |
| | TGFβ-1 anti-apoptotic factor, |
| | HOM-Mel-40/SSX2 |
| Testis | MAGE-1, HOM-Mel-40/SSX2, NY-ESO-1 |
| Colorectal | EGFR, CEA |
| Lung | MAGE D, EGFR |
| Ovarian | Her-2neu |
| Several cancers | NY-ESO-1, glycoprotein MUC1 and |
| | MUC10 mucins, p53 (especially |
| | mutated versions), EGFR |
| Miscellaneous tumor | CDC27 (including the mutated form |
| antigens | of the protein), |
| | triosephosphate isomerase |

In order for tumors to give rise to proliferating and malignant cells, they must become vascularized. Strategies that prevent tumor vascularization have the potential for being therapeutic. The methods and compositions of the present invention can also be used to produce vaccines directed against tumor vascularization. Examples of target antigens for such vaccines are vascular endothelial growth factors, vascular endothelial growth factor receptors, fibroblast growth factors and fibroblast growth factor receptors and the like.

3. Allergen-Related Antigens. The methods and compositions of the present invention can be used to prevent or treat allergies and asthma. According to the present invention, one or more protein allergens can be linked to one or more PAMPs, producing a PAMP/allergen chimeric construct, and administered to subjects that are allergic to that antigen. Thus, the methods and compositions of the present invention can also be used to construct vaccines that may suppress allergic reactions. In this case, the allergen is associated with or combined with a PAMP, including but not limited to BLP, Flagellin or FimC, that can initiate a Th1 response upon binding to a TLR. Initiation of innate immunity via a TLR, for example, tends to be characterized by production and secretion of cytokines, such as IL-12, that elicit a so-called Th1 response in a subject, rather than the typical Th2 response that triggers B-cells to produce immunoglobulin E, the initiator of typical allergic and/or hypersensitive responses. IL-12 produced by dendritic cells and macrophages upon PAMP binding to their TLRs will direct T-cell differentiation into Th1 effector cells. Cytokines produced by Th1 cells, such as Interferon-gamma, will block the differentiation of IL-4 producing Th2 cells and would thus prevent production of antibodies of the IgE isotype, which are responsible for allergic responses.

Specific examples of allergen-related protein antigens useful in the methods and compositions of the present invention include, but are not limited to: allergens derived from pollen, such as those derived from trees such as Japanese cedar (Cryptomeria, Cryptomeria japonica), grasses (Gramineae), such as orchard-grass (Dactylis, Dactylis glomerata), weeds such as ragweed (Ambrosia, Ambrosia artemisiifolia); specific examples of pollen allergens including the Japanese cedar pollen allergens Cry j 1 (J. Allergy Clin. Immunol. (1983)71: 77-86) and Cry j 2 (FEBS Letters (1988) 239: 329-332), and the ragweed allergens Amb a I.1, Amb a I.2, Amb a I.3, Amb a I.4, Amb a II etc.; allergens derived from fungi (Aspergillus, Candida, Alternaria, etc.); allergens derived from mites (allergens from Dermatophagoides pteronyssinus, Dermatophagoides farinae etc.; specific examples of mite allergens including Der p I, Der p II, Der p III, Der p VII, Der f I, Der f II, Der f III, Der f VII etc.); house dust; allergens derived from animal skin debris, feces and hair (for example, the feline allergen Fel d I); allergens derived from insects (such as scaly hair or scale of moths, butterflies, Chironomidae etc., poisons of the Vespidae, such as Vespa mandarinia); food allergens (eggs, milk, meat, seafood, beans, cereals, fruits, nuts and vegetables etc.); allergens derived from parasites (such as roundworm and nematodes, for example, Anisakis); and protein or peptide based drugs (such as insulin). Many of these allergens are commercially available.

In another embodiment, prophylactic treatment of chronic allergies can be accomplished by the administration of a protein PAMP. In a embodiment, the PAMP of the prophylactic vaccine is an OMP, more preferably OspA, and most preferably BLP. Alternatively, Flagellin or FimC can be used as the PAMP.

4. Other Disease Antigens. Also contemplated in this invention are vaccines directed against antigens that are associated with diseases other than cancer, allergy and asthma. As one example of many, and not by limitation, an extracellular accumulation of a protein cleavage product of β-amyloid precursor protein, called “amyloid-β peptide”, is associated with the pathogenesis of Alzheimer's disease. (Janus et al., Nature (2000) 408: 979-982; Morgan et al., Nature (2000) 408: 982-985). Thus, the chimeric construct used in the vaccines of the present invention can include amyloid-β peptide, or antigenic domains of amyloid-β peptide, as the antigenic portion of the construct, and a PAMP or PAMP mimetic. Examples of other diseases in which vaccines might be generated against self proteins or self peptides are shown in the following table.

| Disease | Antigens |
|---|---|
| Autoimmune diseases | disease-linked HLA-alleles (e.g., HLA- |
| | DRB1, HLA-DR1, HLA-DR6 B1 |
| | proteins or fragments thereof, |
| | chain genes); TCR chain sub-groups; |
| | CD11a (leukocyte function-associated |
| | antigen 1; LFA-1); IFNγ; IL-10; TCR |
| | analogs; IgR analogs; 21-hydoxylase (for |
| | Addison's disease); calcium sensing |
| | receptor (for acquired |
| | hypoparathyroidism); tyrosinase (for |
| | vitiligo) |
| Cardiovascular disease | LDL receptor |
| Diabetes | glutamic acid decarboxylase |
| | (GAD); insulin B chain; PC-1; IA-2, |
| | IA-2b; GLIMA-38 |
| Epilepsy | NMDA |

C. PAMPs

PAMPs are discrete molecular structures that are shared by a large group of microorganisms. They are conserved products of microbial metabolism, which are not subject to antigenic variability and are distinct from self-antigens. (Medzhitov et al. (1997) Current Opinion in Immunology 9: 4).

PAMPs can be composed of or found in, but are not limited to, the following types of molecules: Flagellins, chaperones including periplasmic chaperones such as FimC, lipopolysaccharides (LPS), porins, lipid A-associated proteins (LAP), lipopolysaccharides, fimbrial proteins, unmethylated CpG motifs, bacterial DNAs, double-stranded viral RNAs, mannans, cell wall-associated proteins, heat shock proteins, glycoproteins, lipids, cell surface polysaccharides, glycans (e.g., peptidoglycans), phosphatidyl cholines, teichoic acids (e.g., lipoteichoic acids), mycobacterial cell wall components/membranes, bacterial lipoproteins (BLP), outer membrane proteins (OMP), and outer surface protein A (Osp A). (Henderson et al. (1996) Microbiol. Review 60: 316; Medzhitov et al. (1997) Current Opinion in Immunology 9: 4-9).

The preferred PAMPs of the present invention include those that contain a DNA-encoded protein component, such as BLP, Neisseria porin, OMP, Flagellin, FimC and OspA, as these can be used as fusion partners to prepare the preferred embodiment of the invention, i.e., fusion proteins comprising a PAMP and an antigen, preferably a self-antigen. One preferable PAMP for use in the present invention is BLP because BLP is known to induce activation of the innate immune response (Henderson et al. (1996) Microbiol. Review 60: 316) and has been shown to be recognized by TLRs (Aliprantis et al. (1999) Science 285: 763). Flagellin has similarly been demonstrated to induce features of innate immunity (Eaves-Pyles et al., (2001) J. Immunol. 166:1248; Gewirtz et al., (2001) J Clin Invest. 107: 99); Aderem, Presentation at Keystone Symposium, Keystone, Colo.; 2001). As described herein, FimC has been shown to activate NF-κB. Activation of NF-κB is indicative of activation of the Toll-Like Receptors.

Additionally, the present invention contemplates derivatives, portions, parts, or peptides of PAMPs that are recognized by the innate immune system for generating vaccines. As used herein, the terms “fragments of PAMPs”, “portions of PAMPs”, “parts of PAMPs” and “peptides of PAMPs”, all refer to an immunostimulatory part of an entire PAMP molecule. Thus, the PAMPs used in the vaccines of the present invention can comprise an immunostimulatory portion or derivative of the PAMP rather than the entire PAMP, for example E. Coli murein lipoprotein amino acids 1 to 24.

In another embodiment, the present invention contemplates peptide mimetics of non-protein PAMPs. Peptide mimetics of polysaccharides and peptidoglycans are examples of peptide mimetics which can be used in the present invention. The present invention contemplates using phage selection methods to identify peptide mimetics of these non-protein PAMPs. For example, an antibody raised against a non-protein PAMP can be used to screen a phage library containing randomized short-peptide sequences. Selected sequences are isolated and assayed to determine their usefulness as a protein derivative of a non-protein PAMP in the chimeric constructs of the present invention. Such peptide mimetics are useful to produce the recombinant vaccines disclosed herein.

In yet another embodiment, the present invention contemplates further examples of PAMP mimics or PAMP mimetics in which analogs of amino acids and/or peptides are substituted for the amino acid and/or peptide residues, respectively, of a peptide-containing PAMP or a protein PAMP.

In another embodiment, the chimeric construct is a construct comprising CpG or CpG-DNA, and an antigen. The CpG or CpG-DNA can be conjugated to a protein or non-protein antigen. In addition, peptide mimetics of CpG or CpG-DNA, that mimic the structural, functional, antigenic or immunogenic properties of CpG, can be produced and used to generate an antigen-PAMP (where PAMP is a CpG peptide mimetic) protein chimeric construct. This chimeric construct can be produced by recombinant DNA techniques and the expressed fusion protein can be used in the compositions and methods of the present invention.

D. Peptide Mimetics

This invention also includes a mimetic of the three-dimensional structure of a PAMP or antigen. In particular, this invention also includes peptides that closely resemble the three-dimensional structure of non-peptide PAMPs and antigens. Such peptides provide alternatives to non-polypeptide PAMPs or antigens, respectively, by providing the advantages of, for example: more economical production, greater chemical stability, enhanced pharmacological properties (half-life, absorption, potency, efficacy, and/or altered specificity (e.g., a broad-spectrum of biological activities), and other advantages.

Conversely, analogs of PAMP and/or antigen proteins can be synthesized such that one or both consists partially or entirely of amino acid and/or peptide analogs. Such analogs can contain non-naturally-occurring amino acids, or naturally-occurring amino acids that do not commonly occur in proteins, including but not limited to. D-amino acids or amino acids such as β-alanine, omithine or canavanine, and the like, many of which are known in the art. Alternatively, analogs of PAMPs and/or antigens can be synthesized such that one or both consists partially or entirely of peptide analogs containing non-peptide bonds, many examples of which are known in the art. Such analogs may provide greater chemical stability, enhanced pharmacological properties (half-life, absorption, potency, efficacy, etc.) and/or altered specificity (e.g., a broad-spectrum of biological activities) when compared with the naturally-occurring PAMP and/or antigen as well as other advantages.

In one form, the contemplated molecular structures are peptide-containing molecules that mimic elements of protein secondary structure. (see, for example, Johnson et al. (1993) Peptide Turn Mimetics, in Biotechnology and Pharmacy, Pezzuto et al., (editors) Chapman and Hall). Such molecules are expected to permit molecular interactions similar to the natural molecule.

In another form, analogs of peptides are commonly used in the pharmaceutical industry as non-peptide drugs with properties analogous to those of a subject peptide. These types of non-peptide compounds are also referred to as “peptide mimetics” or “peptidomimetics” (Fauchere (1986) Adv. Drug Res. 15, 29-69; Veber et al. (1985) Trends Neurosci. 8: 392-396; Evans et al. (1987) J. Med. Chem. 30: 1229-1239) and are usually developed with the aid of computerized molecular modeling.

Peptide mimetics that are structurally similar to therapeutically useful peptides may be used to produce an equivalent therapeutic or prophylactic effect. Generally, peptide mimetics are structurally similar to a paradigm polypeptide (e.g., a polypeptide that has a biochemical property or pharmacological activity), but have one or more peptide linkages optionally replaced by a linkage selected from the group consisting of: —CH₂NH—, —CH₂S—, —CH₂—CH₂—, —CH═CH— (cis and trans), —COCH₂—, —CH(OH)CH₂—, —CH₂SO— and the like. (Morley (1980) Trends Pharmacol. Sci. 1: 463-468 (general review); Hudson et al. (1979) Int. J. Pept. Protein Res. 14: 177-185 (—CH₂NH—, CH₂CH₂—); Spatola et al. (1986) Life Sci. 38: 1243-1249 (—CH₂—S); Hann (1982) J. Chem. Soc. Perkin Trans. 1: 307-314 (—CH—CH—, cis and trans); Almquist et al. (1980) J. Med. Chem. 23: 1392-1398 (—COCH₂—); Jennings-White et al. (1982) Tetrahedron Lett. 23: 2533 (—COCH₂—); Holladay et al. (1983) Tetrahedron Lett. 24: 4401-4404 (—C(OH)CH₂—); and Hruby (1982) Life Sci. 31: 189-199 (—CH₂S—); each of which is incorporated herein by reference.).

Labeling of peptide mimetics usually involves covalent attachment of one or more labels, directly or through a spacer (e.g., an amide group), to non-interfering position(s) on the peptide mimetic that are predicted by quantitative structure-activity data and molecular modeling. Such non-interfering positions generally are positions that do not form direct contacts with the macromolecule(s) (e.g., in the present example they are not contact points in PAMP-PRR complexes) to which the peptide mimetic binds to produce the therapeutic effect. Derivitization (e.g., labeling) of peptide mimetics should not substantially interfere with the desired biological or pharmacological activity of the peptide mimetic.

PAMP peptide mimetics can be constructed by structure-based drug design through replacement of amino acids by organic moieties. (Hughes (1980) Philos. Trans. R. Soc. Lond. 290: 387-394; Hodgson (1991) Biotechnol. 9: 19-21; Suckling (1991) Sci. Prog. 75: 323-359).

The design of peptide mimetics can be aided by identifying amino acid mutations that increase or decrease binding of PAMP to its PRR. Approaches that can be used include the yeast two-hybrid method (Chien et al. (1991) Proc. Natl. Acad Sci. USA 88: 9578-9582) and using the phage display method. The two-hybrid method detects protein-protein interactions in yeast. (Fields et al. (1989) Nature 340: 245-246). The phage display method detects the interaction between an immobilized protein and a protein that is expressed on the surface of phages such as lambda and M13. (Amberg et al. (1993) Strategies 6: 2-4; Hogrefe et al. (1993) Gene 128: 119-126). These methods allow positive and negative selection for protein-protein interactions and the identification of the sequences that determine these interactions.

Conventional methods of peptide synthesis are known in the art. (Jones (1992) Amino Acid and Peptide Synthesis, Oxford University Press; Jung (1997) Combinatorial Peptide and Nonpeptide Libraries: A Handbook, John Wiley; Bodanszky et al. (1993) Peptide Chemistry—A Practical Textbook, Springer Verlag).

E. Flagellin PAMPs

Bacterial flagella are made up of the structural protein Flagellin, which induces expression of chemokine IL-8 and activation of NF-κB in human and mouse cells. Additionally Flagellin activates mammalian cells via a Toll-Like Receptor, TLR5. These findings, as well as the fact that Flagellin proteins are extremely conserved in bacteria, indicate that Flagellin is a pathogen-associated molecular pattern (PAMP) that would be recognized by the innate immune system.

Because Flagellin is a protein and a PAMP, it is also suitable for the generation of recombinant fusion vaccines. As described in the Examples section below, a series of fusion constructs were tested for their ability to activate the mammalian innate immune system. Activation of NF-κB was used as a read-out in the experiments because it is a critical pathway indicative of the triggering of the Toll-Like Receptors, and has been demonstrated to be a property of the recombinant fusion vaccines.

F. FimC

Gram negative bacteria express filamentous structures called pili on their cell surfaces. Pili are adhesive organelles used by bacteria to interact with host cell surface molecules. Type I pili are made up of several proteins encoded by the fim genes. The assembly of Type I pili and their extrusion through the outer bacterial membrane is highly regulated and requires the presence of a periplasmic protein, FimC. FimC is a conserved periplasmic chaperone of the PapD superfamily. It consists of two immunoglobulin-like domains and participates in the folding and assembly of each pilus subunit in a process called donor strand complementation, which prevents pilus protein aggregation in the periplasm. Because FimC is conserved and absolutely required for pilus assembly, we cloned and expressed it to see if it could induce an innate immune response and potentially be used as an adjuvant for a vaccine.

FimC activated NF-κB in mouse cell lines which express the innate immune system receptors TLR2 and TLR4, suggesting that FimC is a ligand for one of these, or perhaps another Toll-like receptor. Ligation of TLRs is necessary for the induction of an immune response; therefore, FimC is likely to be a good candidate for a vaccine adjuvant. Because FimC is a single gene product, it can be used as a fusion protein with an antigen of interest for the generation of recombinant vaccines.

G. Conservative Variants of PAMPs

The present invention also contemplates conservative variants of naturally-occurring protein PAMPs, peptides of PAMPs, and peptide mime tics of PAMPs that recognize the corresponding PRRs. Such variants are examples of PAMP mimetics. The conservative variations include mutations that substitute one amino acid for another within one of the following groups:
1. Small aliphatic, nonpolar or slightly polar residues: Ala, Ser, Thr, Pro and Gly;
2. Polar, negatively charged residues and their amides: Asp, Asn, Glu and Gln;
3. Polar, positively charged residues: His, Arg and Lys;
4. Large aliphatic, nonpolar residues: Met, Leu, Ile, Val and Cys; and
5. Aromatic residues: Phe, Tyr and Trp.
   The types of substitutions selected may be based on the analysis of the frequencies of amino acid substitutions among the PAMPs of different species (Schulz et al. Principles of Protein Structure, Springer-Verlag, 1978, pp. 14-16) on the analyses of structure-forming potentials developed by Chou and Fasman (Chou et al. (1974) Biochemistry 13: 211; Schulz et al. (1978) Principles in Protein Structure, Springer-Verlag, pp. 108-130), and on the analysis of hydrophobicity patterns in proteins developed by Kyte and Doolittle (Kyte et al. (1982) J. Mol. Biol. 157: 105-132).

The present invention also contemplates conservative variants that do not affect the ability of the PAMP to bind to its PRR. The present invention includes PAMPs with altered overall charge, structure, hydrophobicity/hydrophilicity properties produced by amino acid substitution, insertion, or deletion that retain and/or improve the ability to bind to their receptor. Preferably, the mutated PAMP has at least about 70% sequence identity, more preferably at least about 80% sequence identity, even more preferably, at least about 85% sequence identity, yet more preferably at least about 90% sequence identity, and most preferably at least about 95% sequence identity to its corresponding wild-type PAMP.

Numerous methods for determining percent homology are known in the art. Version 6.0 of the GAP computer program is available from the University of Wisconsin Genetics Computer Group and utilizes the alignment method of Needleman and Wunsch, as revised by Smith and Waterman. (Needleman et al. (1970) J. Mol. Biol. 48: 443; Smith et al. (1981) Adv. Appl. Math. 2: 482). Numerous methods for determining percent identity are also known in the art, and a preferred method is to use the FASTA computer program, which is also available from the University of Wisconsin Genetics Computer Group.

H. Combination Treatments

The present invention provides methods of treating subjects comprising passively immunizing an individual by administering antibodies or activated immune cells to a subject to confer immunity, and administering a vaccine comprising a fusion protein of the present invention, preferably wherein the administered antibody or activated immune cells are directed against the same antigen of the fusion protein of the vaccine. Such treatments can be sequential, in either order or simultaneous. This combination therapy contemplates the use of either monoclonal or polyclonal antibodies that are directed against the antigen of the PAMP/antigen fusion.

The present invention provides methods of treating subjects comprising passively immunizing an individual by administering antibodies or activated immune cells to a subject to confer immunity, and administering a vaccine comprising a chimeric construct of the present invention, wherein the administered antibody or activated immune cells are preferably directed against the same antigen of the chimeric construct. Such treatments can be sequential, in either order, or simultaneous. This combination therapy contemplates the use of either monoclonal or polyclonal antibodies that are directed against the antigen of the PAMP/antigen chimeric construct.

The present invention also contemplates the use of a vaccine comprising a chimeric construct of the present invention in combination with a second treatment where such second treatment is not an immune-directed therapy. A non-limiting example of such combination therapy is the combination of a vaccine comprising a fusion protein of the present invention in combination with a chemotherapeutic agent, such as an anti-cancer chemotherapeutic agent. A further non-limiting example of such combination therapy is the combination of a vaccine comprising a fusion protein construct of the present invention in combination with an anti-angiogenic agent. A further non-limiting example of such combination therapy is the combination of a vaccine comprising a fusion protein of the present invention in combination with radiation therapy, such as an anti-cancer radiation therapy. Yet a further non-limiting example of combination therapy is the combination of a vaccine comprising a fusion protein of the present invention in combination with surgery, such as surgery to remove or reduce vascular blockage.

Also contemplated in this invention is a combination of more than one other therapeutic with a vaccine contemplated in this invention. A non-limiting example is a combination of a vaccine contemplated in this invention in combination with passive immunotherapy treatment and chemotherapy treatment.

In such combination treatments as can be contemplated herein, treatments can be sequential or simultaneous.

The PAMP domain can comprise the entire PAMP or an immunostimulatory portion of the PAMP. Preferably, the fusion protein has maximal immunogenicity and induces minimal inflammatory response. Such desirable properties might be achieved, for example, by using two or more different antigens, and/or portions of different antigens, and/or by using more than one copy of the same antigen or portions of the same antigen, and/or by a combination of both. Alternatively, two or more different PAMPs, or portions of different PAMPs, and/or two or more copies of the same PAMP, or portions of the same PAMP, and/or a combination of both can be used. A further embodiment contemplates fusion proteins containing multiple antigens, and/or portions of antigens, together with multiple PAMPs, and/or portions of PAMPs. It is within the skill of the artisan to determine the desirable ratio of PAMP to antigen domains to maximize immunogenicity and minimize inflammatory response.

There are several advantages of using a fusion system for the production of recombinant polypeptides. First, heterologous proteins and peptides are often degraded by host proteases; this may be avoided, especially for small peptides, by using a gene fusion expression system. Second, general and efficient purification schemes are established for several fusion partners. The use of a fusion partner as an affinity handle allows rapid isolation and purification of the recombinant peptide. Third, by using different fusion partners, the recombinant product may be localized to different compartments or it might be secreted; such strategy could lead to facilitation of purification of the fusion partner and/or directed compartmentalization of the fusion protein.

Additionally, various methods are available for chemical or enzymatic cleavage of the fusion protein that provides efficient strategies to obtain the desired peptide in large quantities. Frequently employed fusion systems include: the Staphylococcal protein A fusion system and the synthetic ZZ variant, both of which have IgG affinity and have been used for the generation of antibodies against short peptides; the glutathione S-transferase fusion system (Smith et al. (1988) Gene 60); the β-galactosidase fusion system; and the trpE fusion system (Yansura (1990) Methods Enzym. 185: 61). Some of these systems are commercially available as kits, including vectors, purification components and detailed instructions.

The present invention also contemplates modified fusion proteins having affinity for metal ion affinity matrices, whereby one or more specific metal-binding or metal-chelating amino acid residues are introduced, by addition, deletion, or substitution, into the fusion protein sequence as a tag. Optimally, a fusion partner, either an antigen or a PAMP domain, is modified to contain an added metal-chelating amino acid tag. The sequence of an antigen or PAMP domain, however, could also be altered to provide a metal-binding site if such modifications could be achieved without adversely affecting a ligand-binding site, an active site, or other functional sites, and/or destroying important tertiary structural relationships in the protein. These metal-binding or metal-chelating residues may be identical or different, and can be selected from the group consisting of cysteine, histidine, aspartate, tyrosine, tryptophan, lysine, and glutamate, and are located so to permit binding or chelation of the expressed fusion protein to a metal. Histidine is the preferred metal-binding residue. The metal-binding/chelating residues are situated with reference to the overall tertiary structure of the fusion protein to maximize binding/chelation to the metal and to minimize interference with the expression of the fusion protein its biological activity.

A fusion sequence of an antigen, PAMP and a tag, may optionally contain a linker peptide. The linker peptide might separate a tag from the antigen sequence or the PAMP sequence. If the linker peptide so used encodes a sequence that is selectively cleavable or digestible by conventional chemical or enzymatic methods, then the tag can be separated from the rest of the fusion protein after purification. For example, the selected cleavage site within the tag may be an enzymatic cleavage site. Examples of suitable enzymatic cleavage sites include sites for cleavage by a proteolytic enzyme, such as enterokinase, Factor Xa, trypsin, collagenase, thrombin and the like. Alternatively, the cleavage site in the linker may be a site capable of cleavage upon exposure to a selected chemical or condition, e.g., cyanogen bromide, hydroxylamine, or low pH, or other chemicals or conditions known in the art.

Cleavage at the selected cleavage site enables separation of the tag from the antigen/PAMP fusion protein. The antigen/PAMP fusion protein may then be obtained in purified form, free from any peptide derivative to which it was previously linked for ease of expression or purification. The cleavage site, if inserted into a linker useful in the fusion sequences of this invention, does not limit this invention. Any desired cleavage site, of which many are known in the art, may be used for this purpose.

Another use of linker peptides might be to direct cleavage of the antigen in intracellular processing so as to facilitate peptide presentation on the surface of the APC. Appropriate cleavage sites might be inserted via linkers such that the fusion protein is not cleaved until it is internalized by the APC. Under such circumstances, such a peptide cleavage site can be introduced via a linker between the PAMP and the antigen to generate intracellular antigen free of PAMP. Such directed cleavage could also be used particularly to facilitate production within the APC of specific peptides that have been identified as interacting with particular HLA haplotypes. Alternatively, different domains from a single antigen or from more than one antigen might be separated by linkers containing cleavage sites so that these epitopes could be appropriately processed for presentation on the surface of the APC.

The optional linker peptide in a fusion protein of the present invention might serve a purpose other than the provision of a cleavage site. As an example, and not by limitation, the linker peptide might be inserted between a PAMP domain and an antigen domain to prevent or alleviate steric hindrance between the two domains. In addition, the linker sequence might provide for post-translational modification including, but not limited to, e.g., phosphorylation sites, biotinylation sites, sulfation sites, carboxylation sites, glycosylation sites, lipidation sites, and the like.

In one embodiment, the fusion protein of this invention contains a domain of an antigen or an immunogenic portion of an antigen fused directly at its amino or carboxyl terminal end to the domain of a PAMP or an immunostimulatory portion of a PAMP. In another embodiment, the fusion protein of this invention contains a domain of a PAMP, or an immunostimulatory portion of a PAMP, or a sequence that can be post-translationally modified to produce a PAMP, inserted within the domain of an antigen, or an immunogenic portion of an antigen. In yet another embodiment, the fusion protein of this invention contains a domain of an antigen, or an immunogenic portion of an antigen, inserted within the domain of a PAMP, or an immunostimulatory portion of a PAMP, or a sequence that can be post-translationally modified to produce a PAMP. In another embodiment, the fusion protein of this invention, comprising an antigen and a PAMP sequence, is fused directly at its amino or carboxyl terminal end to the sequence of a tag. The resulting fusion protein is a soluble cytoplasmic fusion protein. In another embodiment, the fusion sequence further comprises a linker sequence interposed between the antigen sequence and a PAMP sequence or sequence of a tag. This fusion protein is also produced as a soluble cytoplasmic protein.

I. Recombinant Technology

Protein PAMPs, protein antigens, and derivatives thereof can be generated using standard peptide synthesis technology. Alternatively, recombinant methods can be used to generate nucleic acid molecules that encode protein PAMPs, protein antigens and derivatives thereof.

Nucleic acids encoding PAMP/antigen fusions (e.g., synthetic oligo- and polynucleotides) can easily be synthesized by chemical techniques, for example, the phosphotriester method of Matteucci, et al. ((1981) J. Am. Chem. Soc. 103: 3185-3191) or using automated synthesis methods. In addition, larger nucleic acids can readily be prepared by well known methods, such as synthesis of a group of oligonucleotides that define various modular segments of the nucleic acid encoding the PAMP/antigen fusion, followed by ligation of oligonucleotides to build the complete nucleic acid molecule.

The present invention further provides recombinant nucleic acid molecules that encode PAMP/antigen fusion proteins. As used herein, a “recombinant nucleic acid molecule” refers to a nucleic acid molecule that has been subjected to molecular manipulation in vitro. Methods for generating recombinant nucleic acid molecules are well known in the art. (Sambrook et al. (1989) Molecular Cloning: A Laboratory Manual, Cold Spring Harbor Press). In the preferred recombinant nucleic acid molecules, a nucleotide sequence that encodes a PAMP/antigen fusion is operably linked to one or more expression control sequences and/or vector sequences.

The choice of vector and/or expression control sequences to which one of the PAMP/antigen fusion encoding sequences of the present invention is operably linked depends directly, as is well known in the art, on the functional properties desired (e.g., protein expression), and the host cell to be transformed. A vector contemplated by the present invention is at least capable of directing the replication or insertion into the host chromosome, and preferably also expression, of a nucleotide sequence encoding a PAMP/antigen fusion.

Expression control elements that are used for regulating the expression of an operably linked protein encoding sequence are known in the art and include, but are not limited to, inducible promoters, constitutive promoters, secretion signals, enhancers, transcription terminators and other regulatory elements. Preferably, an inducible promoter that is readily controlled, such as being responsive to a nutrient in the medium, is used.

In one embodiment, the vector containing a nucleic acid molecule encoding a PAMP/antigen fusion will include a prokaryotic replicon, e.g., a nucleotide sequence having the ability to direct autonomous replication and maintenance of the recombinant nucleic acid molecule intrachromosomally in a prokaryotic host cell, such as a bacterial host cell, transformed therewith. Such replicons are well known in the art. In addition, vectors that include a prokaryotic replicon may also include a gene whose expression confers a detectable marker such as a drug resistance. Typical bacterial drug resistance genes are those that confer resistance to ampicillin (Amp) or tetracycline (Tet).

Vectors that include a prokaryotic replicon can further include a prokaryotic or viral promoter capable of directing the expression (transcription and translation) of the PAMP/antigen fusion in a bacterial host cell, such as E. coli. A promoter is an expression control element formed by a nucleic acid sequence that permits binding of RNA polymerase and transcription to occur. Promoter sequences compatible with bacterial hosts are typically provided in plasmid vectors containing convenient restriction sites for insertion of a nucleic acid segment of the present invention. Typical of such vector plasmids are pUC8, pUC9, pBR322 and pBR329 available from Biorad Laboratories (Richmond, Calif.), pPL and pKK223 available from Amersham Pharmacia Biotech, Piscataway, N.J.

Expression vectors compatible with eukaryotic cells, preferably those compatible with vertebrate cells, can also be used to express nucleic acid molecules that contain a nucleotide sequence that encodes a PAMP/antigen fusion. Eukaryotic. cell expression vectors are well known in the art and are available from several commercial sources. Typically, such vectors provide convenient restriction sites for insertion of the desired DNA segment. Typical of such vectors are pSVL and pKSV-10 (Amersham Pharmacia Biotech), pBPV-1/pML2d (International Biotechnologies, Inc.), pTDT1 (ATCC, #31255), the vector pCDM8 described herein, and other like eukaryotic expression vectors.

Eukaryotic cell expression vectors used to construct the recombinant molecules of the present invention may further include a selectable marker that is effective in a eukaryotic cell, preferably a drug resistance selection marker. A preferred drug resistance marker is the gene whose expression results in neomycin resistance, e.g., the neomycin phosphotransferase (neo) gene. (Southern et al. (1982) J. Mol. Anal. Genet. 1:327-341). Alternatively, the selectable marker can be present on a separate plasmid, and the two vectors are introduced by cotransfection of the host cell, and selected by culturing in the presence of the appropriate drug for the selectable marker.

The present invention further provides host cells transformed with a nucleic acid molecule that encodes a PAMP/antigen fusion protein of the present invention. The host cell can be either prokaryotic or eukaryotic. Eukaryotic cells useful for expression of a PAMP/antigen fusion protein are not limited, so long as the cell line is compatible with cell culture methods and compatible with the propagation of the expression vector and expression of the fusion protein. Preferred eukaryotic host cells include, but are not limited to, yeast, insect and mammalian cells, preferably vertebrate cells such as those from a mouse, rat, monkey or human fibroblastic cell line.

Any prokaryotic host can be used to express a recombinant nucleic acid molecule. The preferred prokaryotic host is E. coli. In embodiments where the PAMP is a lipoprotein, expression of the PAMP/antigen fusion protein in a bacterial cell is preferred. Expression of the nucleic acid in a bacterial cell line is desirable to ensure proper post-translational modification of the protein portion of the lipoprotein. Preferably, the host cells selected for expression of the PAMP/antigen fusion (e.g. lipoprotein/antigen fusion) is the cell that natively produces the lipoprotein of the lipoprotein/antigen fusion.

Transformation of appropriate cell hosts with nucleic acid molecules encoding a PAMP/antigen fusion of the present invention is accomplished by well known methods that typically depend on the type of vector and host system employed. With regard to transformation of prokaryotic host cells, electroporation and salt treatment methods are typically employed. (See e.g., Cohen et al. (1972) Pro.c Natl. Acad Sci. USA 69:2110; Maniatis et al., Molecular Cloning:. A Laboratory Manual, Cold Spring Harbor Laboratory, Cold Spring Harbor, N.Y. (1982); Sambrook et al. (1989)). With regard to transformation of vertebrate cells with vectors containing rDNAs, electroporation, cationic lipid or salt treatment methods are typically employed. (See e.g., Graham et al., Virology (1973) 52:456;.Wigler et al. (1979) Proc. Natl. Acad. Sci. U.S.A. 76:1373-76).

Successfully transformed cells, e.g., cells that contain a nucleic acid molecule encoding the PAMP/antigen fusions of the present invention, can be identified by well known techniques. For example, cells resulting from the introduction of a nucleic acid molecule encoding the PAMP/antigen fusions of the present invention can be cloned to produce single colonies. Cells from those colonies can be harvested, lysed and their nucleic acids content examined for the presence of the recombinant molecule using a method such as that described by Southern (1975) (J. Mol. Biol. 98: 503), or Berent et al. (1985) (Biotech. 3: 208) or the proteins produced from the cell assayed via an immunological method.

The present invention further provides methods for producing a PAMP/antigen fusion protein that uses one of the nucleic acid molecules herein described. In general terms, the production of a recombinant protein typically involves the following steps.

First, a nucleic acid molecule is obtained that encodes a PAMP/antigen fusion protein. Said nucleic acid molecule is then preferably placed in an operable linkage with suitable control sequences, as described above. The expression unit is used to transform a suitable host and the transformed host is cultured under conditions that allow the production of the PAMP/antigen fusion protein. Optionally, the fusion protein is isolated from the medium or from the cells; recovery and purification of the fusion protein may not be necessary in some instances where some impurities may be tolerated.

Each of the foregoing steps can be done in a variety of ways. For example, the desired coding sequences may be obtained from genomic fragments and used directly in an appropriate host. The construction of expression vectors that are operable in a variety of hosts is accomplished using an appropriate combination of replicons and control sequences. The control sequences expression vectors, and transformation methods are dependent on the type of host cell used to express the gene and were discussed in detail earlier. A skilled artisan can readily adapt any host/expression system known in the art for use with the nucleotide sequences described herein to produce a PAMP/antigen fusion protein.

Endonucleases are nucleases that are able to break internal phosphodiester bonds within a nucleic acid molecule. Examples of nucleases include, but are not limited to, S1 endonuclease from the fungus Aspergillus oryzae, deoxyribonuclease (DNase I), and restriction endonucleases. The cutting and joining processes that underlie DNA manipulation are carried out by enzymes called restriction endonucleases (for cutting) and ligases (for joining). Suitable restriction endonuclease cleavage sites can, if not normally available, be added to the ends of the coding sequence so as to provide an excisable nucleic acid sequence to insert into these vectors.

In addition, restriction endonuclease cleavage sites may also be inserted in the nucleic acid sequence encoding the PAMP/antigen fusion protein. Preferably, these cleavage sites are engineered between nucleotide sequences encoding identical or different PAMPs; between identical or different antigens, or between nucleotide sequences encoding PAMP and antigen. Appropriate cleavage sites well know to those skilled in the art include, but are not limited to, the following: EcoRI, BamHI, BglIII, PvuI, PvuII, HindIII, HinfI, Sau3A, AluI, TaqI, HaeIII and NotI. (T. A. Brown (1996) Gene Cloning: An Introduction, Second Edition, Chapman & Hall, Chapter 4:49-83).

J. Conjugates

The present invention also includes “conjugates” which comprise two or more molecules that are covalently linked, or noncovalently linked but in association with each other. Thus, vaccines of the present invention include PAMP/antigen conjugates such as, but not limited to, the following: protein/nucleic acid conjugates, nucleic acid/protein conjugates, nucleic acid/nucleic acid conjugates, peptide-mimetic/nucleic acid conjugates, nucleic acid/peptide mimetic conjugates, peptide mimetic/peptide mimetic conjugates, lipopolysaccharide/protein conjugates, lipoprotein/protein conjugates, RNA/protein conjugates, CpG-DNA/protein conjugates, nucleic acid analog/protein conjugates, and mannan/protein conjugates. To the extent that PAMPs identified in the future are comprised of yet other chemical classes, conjugates containing such chemicals in combination with antigen domains can also be contemplated.

Methods for the conjugation of polypeptides, carbohydrates, and lipids with DNA are well known to the artisan. See e.g., U.S. Pat. Nos. 4,191,668, 4,650,625, 5,162,515, 5,700,922, 5,786,461, 6,06,0056; and J. Clin. Invest. (1988) 82:1901-1907.

Non-protein PAMPs such as CpG or CpG-DNA, and lipopolysaccharides may be conjugated to protein or non-protein antigens by conventional techniques. For example, PAMP/antigen conjugates may be linked through polymers such as PEG, poly-D-lysine, polyvinyl alcohol, polyvinylpyrollidone, immunoglobulins, and copolymers of D-lysine and D-glutamic acid. Conjugation of the PAMP and antigen to the polymer linker may be achieved in any number of ways, typically involving one or more crosslinking agents and functional groups on the PAMP and antigen. Polypeptide PAMPs and antigens will contain amino acid side chains such as amino, carbonyl, or sulfhydryl groups that will serve as sites for linking the PAMP and antigen to each other. Residues that have such functional groups may be added to either the PAMP or antigen. Such residues may be incorporated by solid phase synthesis techniques or recombinant techniques, both of which are well known in the peptide synthesis arts.

In the case of carbohydrate or lipid analogs, functional amino and sulfhydryl groups may be incorporated therein by conventional chemistry. For instance, primary amino groups may be incorporated by reaction with ethylenediamine in the presence of sodium cyanoborohydride and sulfhydryls may be introduced by reaction of cysteamine dihydrochloride followed by reduction with a standard disulfide reducing agent. In a similar fashion the polymer linker may also be derivatized to contain functional groups if it does not already possess appropriate functional groups. Heterobifunctional crosslinkers, such as sulfosuccinimidyl(4-iodoacetyl) aminobenzoate, which link the epsilon amino group on the D-lysine residues of copolymers of D-lysine and D-glutamate to a sulfhydryl side chain from an amino terminal cysteine residue on the peptide to be coupled, are also useful to increase the ratio PAMPs or antigens in the conjugate.

K. Vaccine Formulation and Delivery

The vaccines of the present invention contain one or more PAMPs, immunostimulatory portions, or immunostimulatory derivatives thereof (e.g., a domain recognized by the innate immune system), and one or more antigens, immunogenic portions, or immunogenic derivatives thereof (e.g., a domain recognized by the adaptive immune system). Since a PAMP mimetic, by definition, has the ability to bind PRRs and initiate an innate immune response, vaccine formulations contemplated by this invention include PAMP mimetics in place of PAMPs. Thus, the present invention contemplates vaccines comprising chimeric constructs including at least one antigen domain and at least one PAMP domain. In one specific embodiment, the vaccines of the present invention comprise a BLP/Eα fusion protein.

The vaccines, comprising the chimeric constructs of the present invention, can be formulated according to known methods for preparing pharmaceutically useful compositions, whereby the chimeric constructs are combined in a mixture with a pharmaceutically acceptable carrier. A composition is said to be a “pharmaceutically acceptable carrier” if its administration can be tolerated by the recipient and if that composition renders the active ingredient(s) accessible at the site where the action is required. Sterile phosphate-buffered saline is one example of a pharmaceutically acceptable carrier. Other suitable carriers are well-known to those in the art. (Ansel et al., Pharmaceutical Dosage Forms and Drug Delivery Systems, 5^{th} Edition (Lea & Febiger 1990); Gennaro (ed.), Remington's Pharmaceutical Sciences 18th Edition (Mack Publishing Company 1990)).

Examples of several other excipients that can be contemplated may include, water, dextrose, glycerol, ethanol, and combinations thereof. The vaccines of the present invention may further contain auxiliary substances, such as wetting or emulsifying agents, pH buffering agents, stabilizers or other carriers that include, but are not limited to, agents such as aluminum hydroxide or phosphate (alum), commonly used as a 0.05 to 0.1 percent solution in phosphate buffered saline, to enhance the effectiveness thereof.

The chimeric constructs of the present invention can be used as vaccines by conjugating to soluble immunogenic carrier molecules. Suitable carrier molecules include protein, including keyhole limpet hemocyanin, which is a preferred carrier protein. The chimeric construct can be conjugated to the carrier molecule using standard methods. (Hancock et al., “Synthesis of Peptides for Use as Immunogens,” in Methods in Molecular Biology: Immunochemical Protocols, Manson (ed.), pages 23-32 (Humana Press 1992)).

Furthermore, the present invention contemplates a vaccine composition comprising a pharmaceutically acceptable injectable vehicle. The vaccines of the present invention may be administered in conventional vehicles with or without other standard carriers, in the form of injectable solutions or suspensions. The added carriers might be selected from agents that elevate total immune response in the course of the immunization procedure.

Liposomes have been suggested as suitable carriers. The insoluble salts of aluminum, that is aluminum phosphate or aluminum hydroxide, have been utilized as carriers in routine clinical applications in humans. Polynucleotides and polyelectrolytes and water soluble carriers such as muramyl dipeptides have been used.

Preparation of injectable vaccines of the present invention, includes mixing the chimeric construct with muramyl dipeptides or other carriers. The resultant mixture may be emulsified in a mannide monooleate/squalene or squalane vehicle. Four parts by volume of squalene and/or squalane are used per part by volume of mannide monooleate. Methods of formulating vaccine compositions are well-known to those of ordinary skill in the art. (Rola, Immunizing Agents and Diagnostic Skin Antigens. In: Remington's Pharmaceutical Sciences, 18th, Edition, Gennaro (ed.), (Mack Publishing Company 1990) pages 1389-1404).

Additional pharmaceutical carriers may be employed to control the duration of action of a vaccine in a therapeutic application. Control release preparations can be prepared through the use of polymers to complex or adsorb chimeric construct. For example, biocompatible polymers include matrices of poly(ethylene-co-vinyl acetate) and matrices of a polyanhydride copolymer of a stearic acid dimer and sebacic acid. (Sherwood et al. (1992) Bio/Technology 10: 1446). The rate of release of the chimeric construct from such a matrix depends upon the molecular weight of the construct, the amount of the construct within the matrix, and the size of dispersed particles. (Saltzman et al. (1989) Biophys. J. 55: 163; Sherwood et al., supra.; Ansel et al. Pharmaceutical Dosage Forms and Drug Delivery Systems, 5th Edition (Lea & Febiger 1990); and Gennaro (ed.), Remington's Pharmaceutical Sciences, 18th Edition (Mack Publishing Company 1990)). The chimeric construct can also be conjugated to polyethylene glycol (PEG) to improve stability and extend bioavailability times (e.g., Katre et al.; U.S. Pat. No. 4,766,106).

The vaccines of this invention may be administered parenterally. The usual modes of administration of the vaccine are intramuscular, sub-cutaneous, and intra-peritoneal injections. Moreover, the administration may be by continuous infusion or by single or multiple boluses.

The gene gun has also been used to successfully deliver plasmid DNA for inducing immunity against an intracellular pathogen for which protection primarily depends on type 1 CD8.sup. +T-cells. (Kaufmann et al. (1999) J. Immun. 163(8): 4510-4518).

Gene transfer-mediated vaccination methods have become a rapidly expanding field and the compositions of the present invention are applicable to the treatment of both noninfectious and infectious diseases and noninfectious diseases, including but not limited to genetic disorders, using such vaccination methods. (See e.g., Eck et al. (1996) Gene-Based Therapy, In: Goodman & Gilman's The Pharmacological Basis of Therapeutics, Ninth Edition, Chapter 5, McGraw Hill).

Alternatively, the vaccine of the present invention, particularly as regards use of Flagellin as a PAMP, may be formulated and delivered in a manner designed to evoke an immune response at a mucosal surface. Thus, the vaccine compositions may be administered to mucosal surfaces by, for example, nasal or oral (intragastric) routes. Other modes of administration include suppositories and oral formulations. For suppositories, binders and carriers may include polyalkalene glycols or triglycerides. Oral formulations may include normally employed incipients such as pharmaceutical grades of saccharine, cellulose and magnesium carbonate. These compositions can take the form of solutions, suspensions, tablets, pills, capsules, sustained release formulations or powders and contain about 1 to 95% of the chimeric construct. The vaccines are administered in a manner compatible with the dosage formulation, and in such amount as will be therapeutically effective, protective and immunogenic dosages.

The quantity of vaccine employed will of course vary depending upon the patient's age, weight, height, sex, general medical condition, previous medical history, the condition being treated and its severity, and the capacity of the individual's immune system to synthesize antibodies, and produce a cell-mediated immune response. Typically, it is desirable to provide the recipient with a dosage of the chimeric construct which is in the range of from about 1: g agent/kg body weight of patient to 100 mg agent/kg body weight of patient, although a lower or higher dosage may also be administered. Precise quantities of the active ingredient, however, depend on the judgment of the practitioner. Suitable dosage ranges are readily determinable by one skilled in the art and may be on the order of nanograms of the chimeric construct to grams of the chimeric construct, depending on the particular construct. Preferably the dosage range of the active ingredient is nanograms to micrograms; more preferably nanograms to milligrams; and most preferably micrograms to milligrams. Suitable regimes for initial administration and booster doses are also variable, but may include an initial administration followed by subsequent administrations. The dosage may depend on the route of administration and will vary according to the size of the subject.

The present invention encompasses vaccines containing antigen and PAMPs from a single organism, such as from a specific pathogen. The present invention also encompasses vaccines that contain antigenic material from several different sources and/or PAMP material isolated from several different sources. Such combined vaccines contain, for example, antigen and PAMPs from various microorganisms or from various strains of the same microorganism, or from combinations of various microorganisms.

For purposes of therapy, the antigen/PAMP fusion proteins are administered to a mammal in a therapeutically effective amount. A vaccine preparation is said to be administered in a “therapeutically effective amount” if the amount administered is can produce a measurable positive effect in a recipient. In particular, a vaccine preparation of the present invention produces a positive effect in a recipient if it invokes a measurable humoral and/or cellular immune response in the recipient. In particular, this invention contemplates a desirable therapeutically effective amount as one in which the vaccine invokes in the recipient a measurable humoral and/or cellular immune response versus the target antigen but causes neither excessive non-specific inflammation nor an autoimmune response versus non-target antigen(s).

As used herein, the term “treatment” refers to both therapeutic treatment and prophylactic or preventative treatment. In one embodiment, the present invention contemplates using the disclosed vaccines to treat patients in need thereof. The patients may be suffering from diseases such as, but not limited to, cancer, allergy, infectious disease, autoimmune disease, neurological disease, cardiovascular disease, or a disease associated with an allergic reaction. In another embodiment, the present invention contemplates administering the disclosed vaccines to passively immunize patients against diseases such as but not limited to, cancer, allergy, infectious disease, autoimmune disease, neurological disease, cardiovascular disease, or disease associated with an allergic reaction. In yet another embodiment the present invention contemplates administering the disclosed vaccines to immunize patients against diseases in addition to those cited in the previous sentence in which the objective is to rid the body of specific molecules or specific cells. A non-limiting example might be the removal or prevention of deposition of plaque in cardiovascular disease.

L. Treatment/Enhancement of Immunity

The vaccines of the present invention can be used to enhance the immunity of animals, more specifically mammals, and even more specifically humans (e.g., patients) in need thereof. Enhancement of immunity is a desirable goal in the treatment of patients diagnosed with, for example, cancer, immune deficiency syndrome, certain topical and systemic infections, leprosy, tuberculosis, shingles, warts, herpes, malaria, gingivitis, and atherosclerosis.

The advantages of the vaccines of the present invention are that they induce a strong immune response against the target antigen with minimal undesired inflammatory reaction, as well as minimal instances of autoimmune disease. Such a reduced side effect profile has a distinct advantage over other vaccine approaches, particularly with respect to targeting of self antigens, because with many other vaccine strategies, in order to elicit a robust response against the self antigen, strong adjuvants are used and they result in excessive inflammation and can increase the risk of autoimmune disease.

As used herein, “immunoenhancement” refers to any increase in an organism's capacity to respond to foreign antigens or other targeted antigens, such as those associated with cancer, which includes an increased number of immune cells, increased activity and increased ability to detect and destroy such antigens, in those cells primed to attack such antigens.

The strength of an immune response can be measured by standard tests including, but not limited to, the following: direct measurement of peripheral blood lymphocytes by means known to the art; natural killer cell cytotoxicity assays (Provinciali et al. (1992) J. Immunol. Meth. 155: 19-24), cell proliferation assays (Vollenweider et al. (1992) J. Immunol. Meth. 149: 133-135), immunoassays of immune cells and subsets (Loeffler et al. (1992) Cytom. 13: 169-174; Rivoltini et al. (1992) Can. Immunol. Immunother. 34: 241-251); and skin tests for cell-mediated immunity (Chang et al. (1993) Cancer Res. 53: 1043-1050). For an excellent text on methods and analyses for measuring the strength of the immune system, see, for example, Coligan et al. (Ed.) (2000) Current Protocals in Immunology, Vol. 1, Wiley & Sons.

Any statistically significant increase in the strength of immune response, as measured by the above tests, is considered “enhanced immune response” or “immunoenhancement”. An increase in T-cells in S-phase of greater than 5 percent has been achieved by the methods of this invention. Enhanced immune response is also indicated by physical manifestations such as fever and inflammation, although one or both of these manifestations might not be observed with the recombinant vaccines of the present invention. Enhanced immune response is also characterized by healing of systemic and local infections, and reduction of symptoms in disease, e.g. decrease in tumor size, alleviation of symptoms of leprosy, tuberculosis, malaria, naphthous ulcers, herpetic and papillomatous warts, gingivitis, atherosclerosis, the concomitants of AIDS such as Kaposi's sarcoma, bronchial infections, and the like.

M. Vaccine Production

The procedures of the present invention can be used to generate a chimeric construct comprising one or more antigens of interest and one or more PAMPs. A small, non-immunogenic epitope tag (such as a His tag) can be added to facilitate the purification of fusion protein expressed in bacteria. The combination of antigen with a PAMP such as BLP, Flagellin or FimC provides signals necessary for the activation of the antigen-specific adaptive and innate immune responses.

A large number of differing fusion proteins comprising different combinations of antigens and PAMPs can be readily generated using recombinant DNA technology or conjugation chemistry that is well known in the art. Virtually any antigen can be used to generate a vaccine by this approach using the same technology. This novel approach, therefore, is very versatile.

Large amounts of recombinant vaccine product can be generated using a bacterial expression system. The product can be purified from bacterial cultures using standard techniques. The approach is thus extremely economical and cost efficient. Alternatively, recombinant vaccine product can be produced and purified from cultures of yeast or other eukaryotic cells including, without limitation, insect cells or mammalian cells. Conjugated non-protein vaccine product can also be produced chemically in relatively large amounts. This is particularly the case if the PAMP and the antigen can both be obtained by relatively straightforward purification procedures and then conjugated together with relatively simple and efficient conjugation chemistry.

Alternatively, a chimeric construct containing a protein component and a non-protein component can be conveniently obtained by preparing the protein component by recombinant means and the non-protein component by chemical means and then linking the two components with linker chemistry well known in the art, some of which is described herein. Additionally, since the antigens and PAMPs contemplated in this invention can be naturally occurring, they can be purified from their natural sources and then linked together chemically. Both T-cell and B-cell antigens can be used to generate vaccines by this approach.

Fusion of an antigen with a PAMP such as BLP, Flagellin or FimC optimizes the stoichiometry of the two signals thus minimizing the unwanted excessive inflammatory responses (which occur, for example, when antigens are mixed with adjuvants to increase their immunogenicity).

Fusion of an antigen with a PAMP such as BLP increases the likelihood that APCs activated in response to the vaccine productively trigger the desired adaptive immune response. Activation of such APCs in the absence of uptake and presentation of the antigen can lead to the induction of autoimmune responses, which, again, is one of the problems with commonly used adjuvants that prevents or limits their use in humans.

In a preferred embodiment, the fusion proteins of the present invention comprise an antigen or an immunogenic portion thereof which has been modified to contain an amino acid sequence comprising a leader sequence and a consensus sequence, that results in the post-translational modification of the consensus sequence or a portion of that sequence, wherein the post-translationally modified sequence is a ligand for a PRR. The modified antigens include, but are not limited to, antigens that contain the bacterial lipidation consensus sequence CXXN (SEQ ID NO: 1), wherein X is any amino acid, but preferably serine. Numerous leader sequences are well known in the art, but a preferred leader sequence is described by the first 20 amino acids of SEQ ID NO: 2, wherein the first 20 amino acids of SEQ ID NO: 2 are set forth in set forth in SEQ ID NO: 3. Examples of additional suitable leader sequences are described in the Sequence Listing as SEQ ID NO: 4-7. A preferred chimeric construct comprises a leader sequence fused, in frame, to a sequence comprising the bacterial lipidation consensus sequence of SEQ ID NO: 1 further fused to an antigen (e.g. leader sequence-CXXN-antigen). Although this modification of the antigen can be referred to as a fusion, this modification can be achieved without fusing DNA, but rather by introducing, by mutagenesis, a leader sequence followed by the CXX sequence into DNA encoding any antigen of interest. Expression of a nucleic acid molecule encoding this chimeric construct, in a bacterial host cell, produces a substrate, first for bacterial proteases, that cleave the leader sequence from the modified antigen, and bacterial lipid transferases, which lipidate the sequence, or a portion thereof, comprising the lipidation consensus sequence. The resultant product is a chimeric construct or fusion protein that is a ligand for a PRR and is capable of stimulating both the innate and adaptive immune systems. In an additional embodiment, this chimeric construct or fusion protein comprises additional polar or charged amino acids to increase the hydrophilicity of the chimeric construct or fusion protein without altering the immunogenic or immunostimulatory properties of the construct.

Without further description, it is believed that one of ordinary skill in the art can, using the preceding description and the following illustrative examples, practice the methods of the present invention. The following working examples, therefore, specifically point out embodiments of the present invention, are illustrative only, and are not to be construed as limiting in any way the remainder of the disclosure. Other generic and specific configurations will be apparent to those persons skilled in the art.

### EXAMPLES

### Example 1

### Model Vaccine Cassette with an Antigen Domain and a PAMP Domain

In order to produce a model vaccine cassette of the present invention, we fused a pathogen-associated molecular pattern (PAMP) to the characterized mouse antigen, Eα. The PAMP we selected, BLP, is known to stimulate innate immune responses through the receptor, Toll-like-receptor-2 (TLR-2).

The protein sequence of the bacterial lipoprotein (BLP) used in the vaccine cassette for fusion with an antigen of interest is as follows:
MKATKLVLGAVILGSTLLAGCSSNAKIDQLSSDVQTLNAKVDQLSNDVNAM RSDVQAAKDDAARANQRLDNMATKYRK (SEQ ID NO: 2). The leader sequence includes amino acid number 1 through amino acid number 20 of SEQ ID NO: 2. The first cysteine (amino acid number 21 of SEQ ID NO: 2) is lipidated in bacteria. This lipidation, which can only occur in bacteria, is essential for BLP recognition by Toll and TLRs. The C-terminal lysine (amino acid number 78 of SEQ ID NO: 2) was mutated to increase the yield of a recombinant vaccine, because this lysine can form a covalent bond with the peptidoglycan.

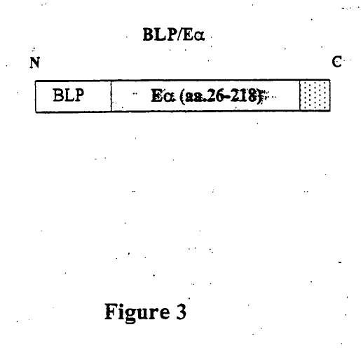

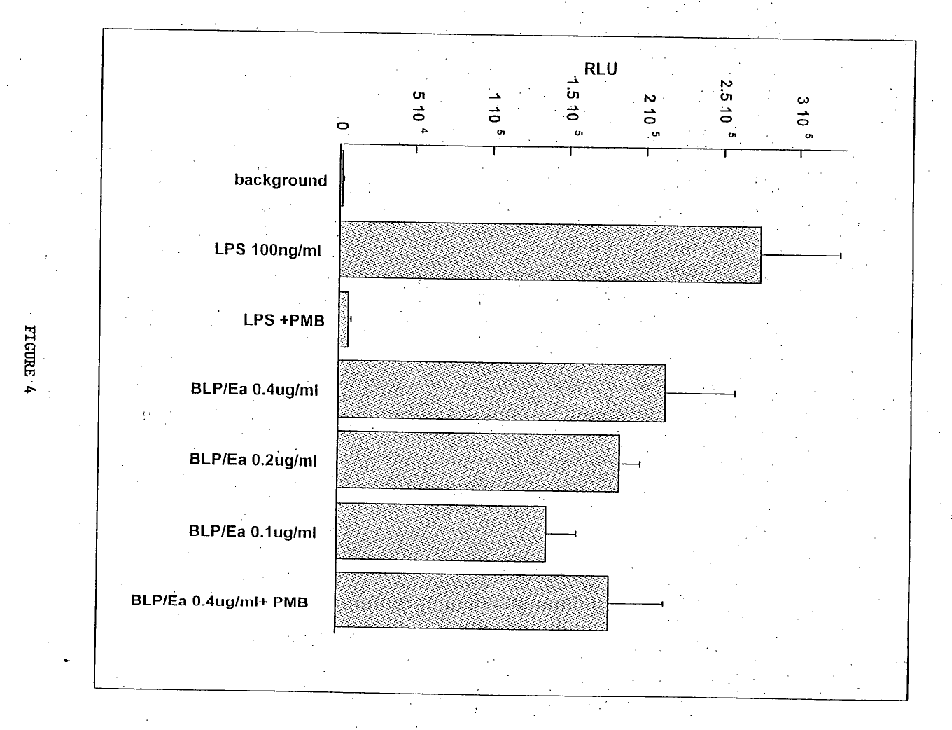

To assist in identification and purification of the antigen, a hexa-histidine tag was engineered on the C-terminal of the protein. The final construct is shown in FIG. 3.

The fusion protein was expressed in bacteria and induced with IPTG. The protein was purified by lysis and sonication in 8 M Urea, 20 mM Tris, 20 mM NaCl, 2% Triton-X-100, pH 8.0. The lysate was passed over a 100 ml Q-Sepharose ion exchange column in the same buffer and washed with 5 column volumes of 8 M Urea, 20 mM Tris, 20 mM NaCl, 0.2% Triton-X-100, pH 8.0. The protein was eluted by salt gradient (20 mM NaCl to 800 mM NaCl). Positive fractions were identified by immunoblotting using an antibody to the Histidine tag. These fractions were pooled and passed over a 2 ml nickel-agarose column. The column was extensively washed with the same buffer (10 column volumes) and then washed with 5 column volumes of phosphate buffer (20 mM) containing 200 mM NaCl, 0.2% Triton-X-100, 20 mM imidazole, pH 8.0. The purified protein was eluted in 20 mM phosphate buffer, 200 mM NaCl, 0.1% Triton-X-100, 250 mM imidazole and fractions were again tested for protein by immunoblotting. Positive fractions were pooled and dialyzed overnight against phosphate buffered saline containing 0.1% Triton-X-100. The sample was then decontaminated of any endotoxin by passage over a polymyxin B column, and concentrated in an Amicon concentrator by centrifugation and tested by immunoblotting and protein concentration for protein content.

### Example 2

### Stimulation of NF-κB by BLP/Eα Model Antigen in RAW Cells

To test whether the model antigen could stimulate signal transduction pathways necessary for an immune response, we assayed NF-κB activation in the RAW mouse macrophage cell line in vitro. We developed a stable RAW cell line that harbors an NF-κB-dependent firefly luciferase gene. Stimulation of these cells with activators of NF-κB leads to production of luciferase which is measured in cell lysates by use of a luminometer. Cells were stimulated with the indicated amounts of BLP/Eα, left 5 hours and harvested for luciferase measurement.

As a control, RAW cells were stimulated with LPS in the presence and absence of polymyxin B (PmB). PmB inactivates endotoxin and as expected the activation of NF-κB activity in the LPS+PmB sample is diminished by 98%. BLP/Eα also activates NF-κB in a dose-dependent manner as shown in FIG. 4, however, treatment with PmB does not inactivate the stimulus to a statistically significant degree. These results suggest that the activation of NF-κB seen with BLP/Eα is not due to contamination of the preparation with endotoxin.

### Example 3

### BLP/Eα Model Vaccine Induces the Production of IL-6 by Dendritic Cells in Vitro

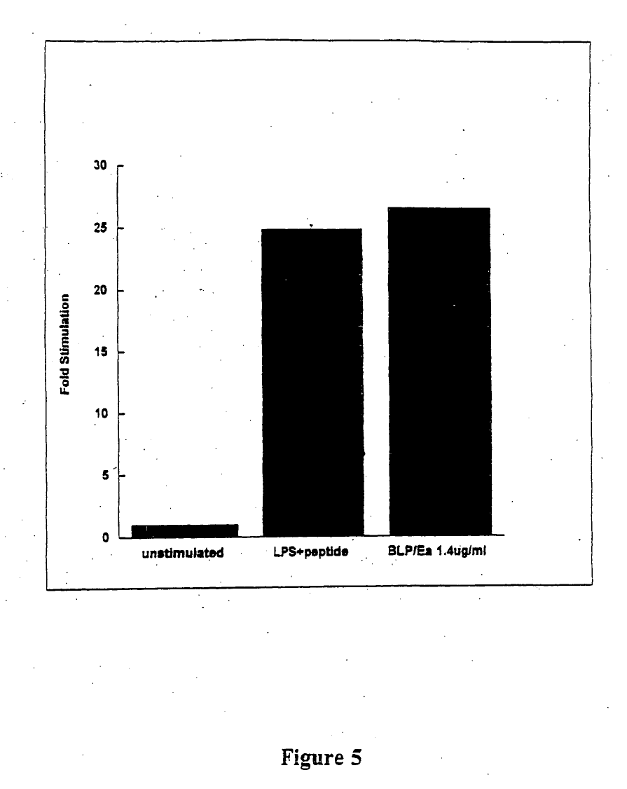

An effective vaccine must be able to stimulate dendritic cells (DC)to mature and present antigen. To test whether BLP/Eα could induce DC function, we tested the ability of bone marrow-derived DC to produce IL-6 after stimulation in vitro. Bone marrow dendritic cells were isolated and grown for 5 days in culture in the presence of 1% GM-CSF. After 5 days, cells were replated at 250,000 cells/well in a 96-well dish and treated with either Eα peptide (0.3: g/ml), LPS (100 ng/ml) +Eα peptide (0.3: g/ml), or BLP/Eα. BLP/Eα was able to stimulate IL-6 production in these cells as measured in a sandwich ELISA (FIG. 5).

### Example 4

### BLP/Eα Stimulates Maturation of Immature Dendritic Cells

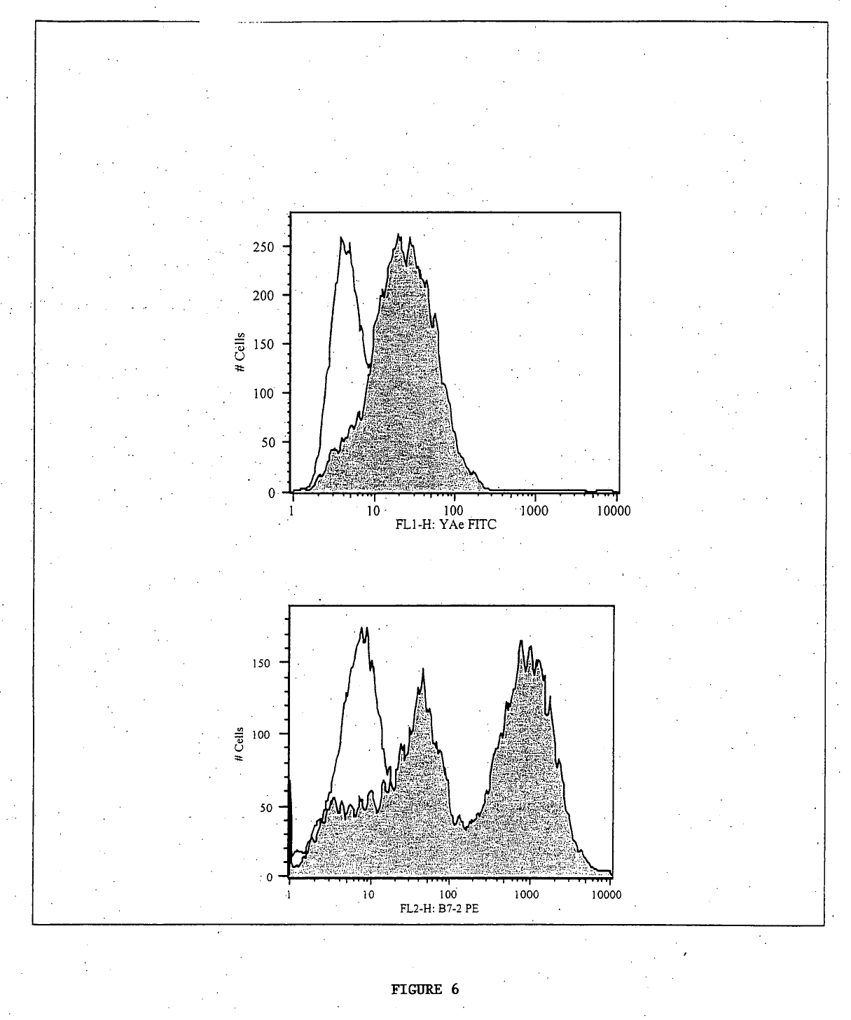

To determine whether BLP/Eα vaccine can be processed and presented by dendritic cells, we stimulated dendritic cells with the vaccine and tested them for the surface expression of B7.2 and Eα peptide bound to MHC Class II. Cultured bone marrow-derived dendritic cells (5 days) were stimulated with Eα peptide or BLP/Eα and were stained with an antibody to the B7.2 costimulatory molecule and/or with Yae antibody which recognizes Eα peptide bound to MHC Class II. Analysis was performed by FACS (FIG. 6).

### Example 5

### BLP/Eα Model Vaccine Stimulates Specific T-Cells In Vitro

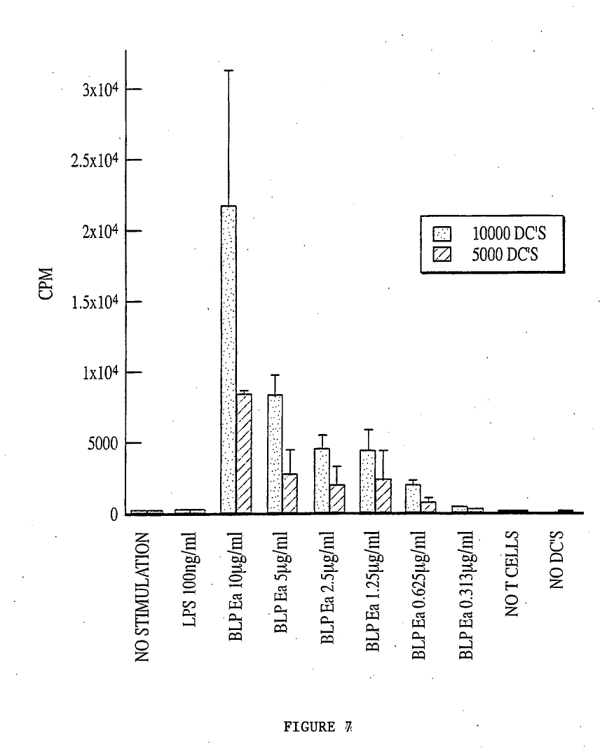

We next assayed whether BLP/Eα that was processed and presented by DC could stimulate the proliferation of antigen-specific T-cells in vitro. Bone marrow derived mouse DC were isolated and plated into medium containing 1% GM-CSF at 750,000 cells/well. Cells were cultured for 6 days and then the DC were collected, washed, and counted then replated in 96-well dishes at 250,000 cells per well. Cells were stimulated with the above indicated antigens and left three days to mature. After 3 days, the DC were resuspended and plated in a 96-well dish at either 5,000 or 10,000 cells/well. T-cells from lymph nodes from a 1H3.1 TCR transgenic mouse (1H3.1 TCR is specific for the Eα peptide) were plated on the DC at 100,000 cells/well. Cells were left for 3 days in culture then “pulsed” with 0.5: Ci/well of ³H-thymidine. The cells were harvested 24 hours later and incorporation of thymidine (T-cell proliferation) was measured in cpm (FIG. 7).

### Example 6

### BLP/Eα Activates Specific T-cells In Vivo

To assess the ability of the vaccine to generate a specific T-cell response in vivo, we injected the fusion protein into a mouse. Three mice were injected as follows:

| Mouse # | Sample injected | # of lymph node cells |
|---|---|---|
| 1 | Eα peptide 30: g in PBS | 1.9 × 10⁶ |
| 2 | Eα peptide 30: g in CFA* | 3.29 × 10⁷ |
| 3 | BLP/Eα 100: g | 5.2 × 10⁶ |

*Complete Freund's Adjuvant

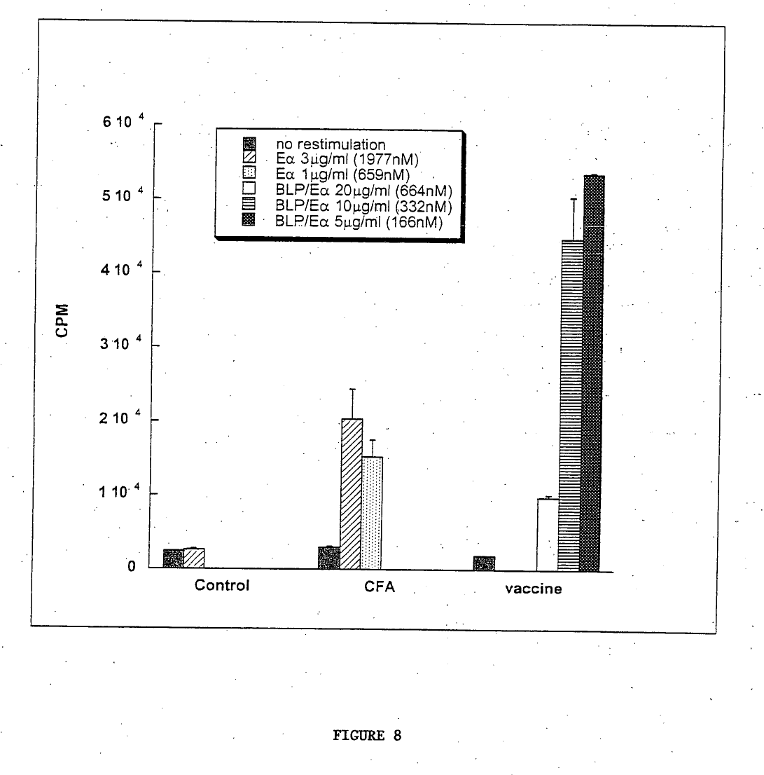

The injected footpad of mouse #2 was considerably swollen for the duration of the experiment, but the footpads of mice #1 and #3 appeared normal. After 6 days, the mice were euthanized and the associated draining lymph node was harvested for a T-cell proliferation assay. T-cells were plated in a 96-well plate at 400,000 cells/well and were restimulated with either Eα peptide or with BLP/Eα at the indicated doses. Cells were left 48 hours to begin proliferation, pulsed with 0.5: Ci/well of³H-Thymidine in medium and harvested 16 hours later. Thymidine incorporation was measured by counting in a beta-plate reader (FIG. 8).

### Example 7

### Model Vaccine Cassette with an Allergen-Related Antigen

Using the procedures set forth above for the production of the BLP/Eα model antigen, a vaccine cassette with an allergen-related antigen is produced using the pollen allergen Ra5G from the giant ragweed (Ambrosia trifida). The amino acid sequence of Ra5G is as follows:
MKNIFMLTLF ILIITSTIKA IGSTNEVDEI KQEDDGLCYE GTNCGKVGKY CCSPIGKYCVCYDSKAICNK NCT (SEQ ID NO: 9).

The amino acid sequence of this allergen can be fused with the BLP amino acid sequence (SEQ ID NO: 1) to generate the BLP/Ra5G fusion protein. The resultant recombinant vaccine places the allergen in the context of an IL-12 inducing signal, where the PAMP in this case is BLP).

When introduced into a subject, this vaccine will generate allergen-specific T-cell responses that will be differentiated into Th1 responses due to the induction of IL-12 by BLP in dendritic cells and macrophages.

### Example 8

### Model Vaccine Cassette with a Tumor-Related Antigen

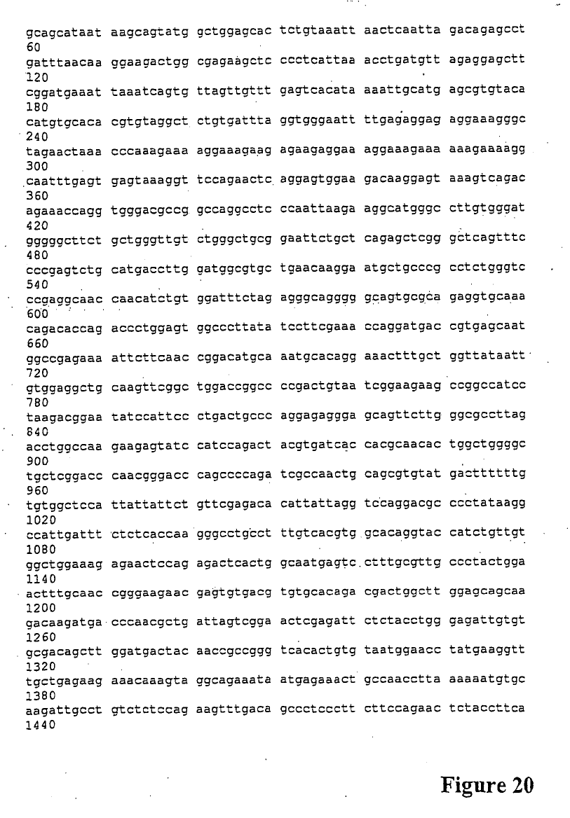

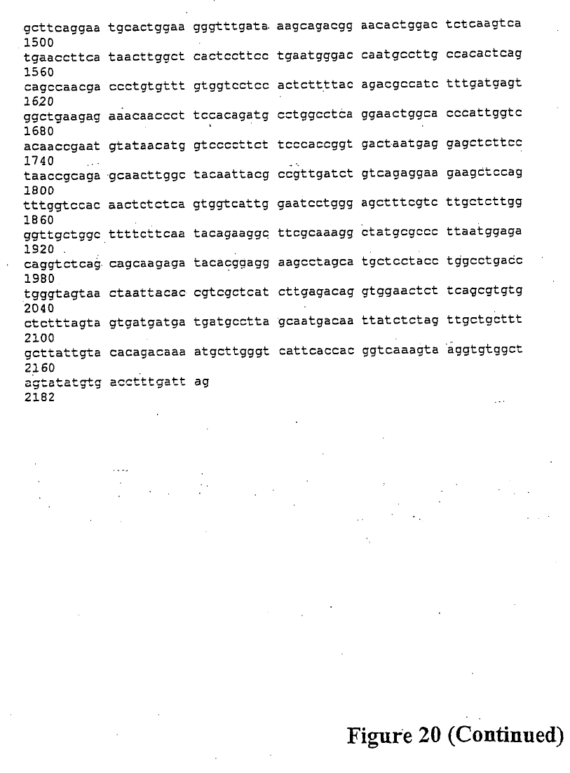

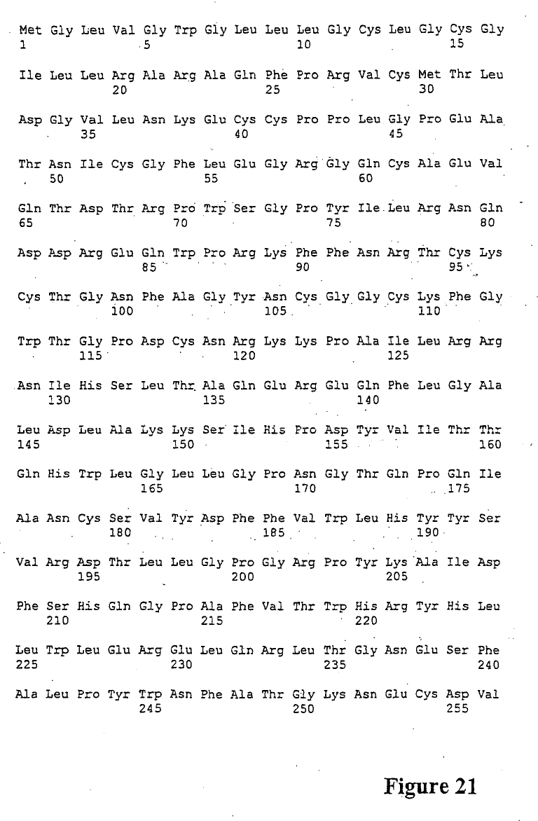

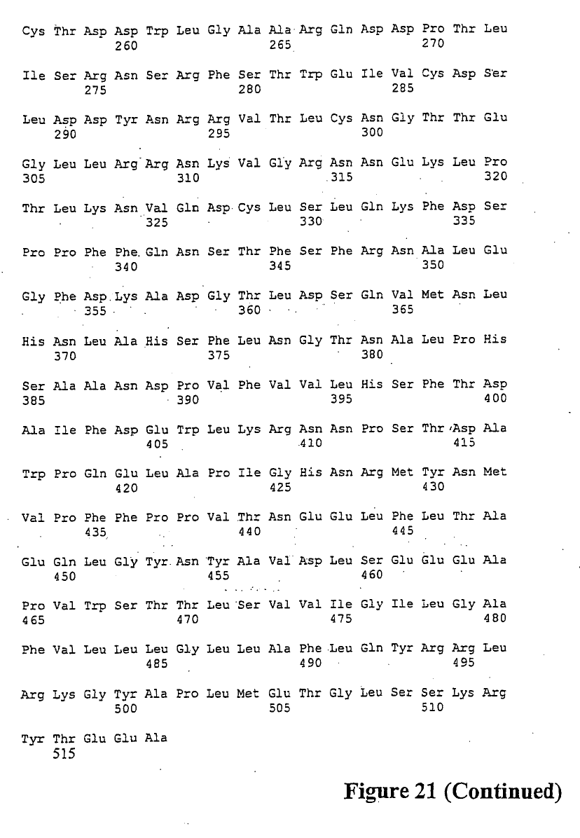

Using the procedures set forth above for the production of the BLP/Eα model antigen, a vaccine cassette with a tumor-related antigen is produced using the model tumor antigen, Tyrosinase-Related Protein 2 (TRP-2). The nucleic acid sequence and corresponding amino acid sequence of TRP-2 is provided in SEQ ID NO: 10 (shown in FIG. 20) and SEQ ID NO: 11 (shown in FIG. 21), respectively. The region used for BLP fusion includes nucleic acid number 840 through nucleic acid number 1040 of SEQ ID NO: 10. The T-cell epitope includes nucleic acid number 945 through nucleic acid number 968 of SEQ ID NO: 10.

A region of the TRP-2 that can be used for the vaccine construction is shown below:

(SEQ ID NO: 12)

LDLAKKSIHPDYVITTQHWLGLLGPNGTQPQIANCSVYDFFVWLHYYSVR
DTLLGPGRPYKAIDFSHQ.

A T-cell epitope of SEQ ID NO: 12 is VYDFFVWL (SEQ ID NO: 13).

### Example 9

### CpG Immunostimulation

The family of TLRs has recently been identified as an essential component of innate immune recognition in both Drosophila and mammalian organisms (Hoffmann et al. (1999) Science 284:1313-1318; Imler et al. (2000) Curr. Opin. Microbiol. 3:16-22). Drosophila Toll is required for the detection of fungal infection and the induction of the antifungal peptide drosomycin (Lemaitre et al. (1996) Cell 86:973-983). In the mouse, TLR2 and TLR4 were shown to mediate recognition of bacterial PGN and LPS, respectively (Takeuchi et al. (1999) Immunity 11:443-451). The functions of the other members of the Drosophila and mammalian Toll families are currently unknown, although it is expected that at least some of them are involved in innate immune recognition as well.

Collectively, the results described here indicate that the immunostimulatory effect of CPG-DNA on the three types of professional antigen presenting cells-DC, macrophages and B-cells—is mediated by a MyD88 signaling pathway. MyD88 is involved in signal transduction by the Toll and IL-1 receptor families. The activities of the IL-1 family of cytokines, including IL-1 and IL-18, is dependent on processing by caspase-1, but in all the experiments described here, the absence of caspase-1 had no effect on CpG-DNA induced cellular responses (Fantuzzi et al. (1999) J. Clin. Immunol. 19:1-11).

We tested whether TLR2 and TLR4 are involved in the recognition of CpG-DNA and found that they are not, at least based on the assays provided herein. We believe, therefore, that CpG-DNA is recognized by a Toll receptor other than TLR2 and TLR4. Cell lines that express endogenous or transfected TLR1 through TLR6 did not respond to CpG-DNA (data not shown), suggesting that some other member of the Toll family may mediate CpG-DNA recognition.

While the identity of the Toll receptor that is responsible for CpG-DNA recognition remains unknown at this point, the fact that CpG-DNA requires internalization to exert its stimulatory effect (Krieg et al. (1995) Nature 374:546-549; Stacey et al. (1996) J. Immunol. 157:2116-2122) suggests that the TLR that mediates the recognition may be expressed in an intracellular compartment, such as the late endosome, phagosome, or lysosome.

### Example 10

### CpG and B-Cell Activation

B-cells from the indicated mouse strains were purified from spleen by complement kill of CD4⁺, CD8⁺ and macrophages. Non-adherent cells were cultured in the presence or absence of different amounts of stimulating CpG-DNA (5′-TCCATGACGTTCCTGACGTT-3′ (SEQ ID NO. 8), phosphorothioate modified) at 1×10⁶ cells/ml. After 48 h, the cells were pulsed with [³H]thymidine (0.5 μCi per well, NEN) for 16 h and processed for beta counting.

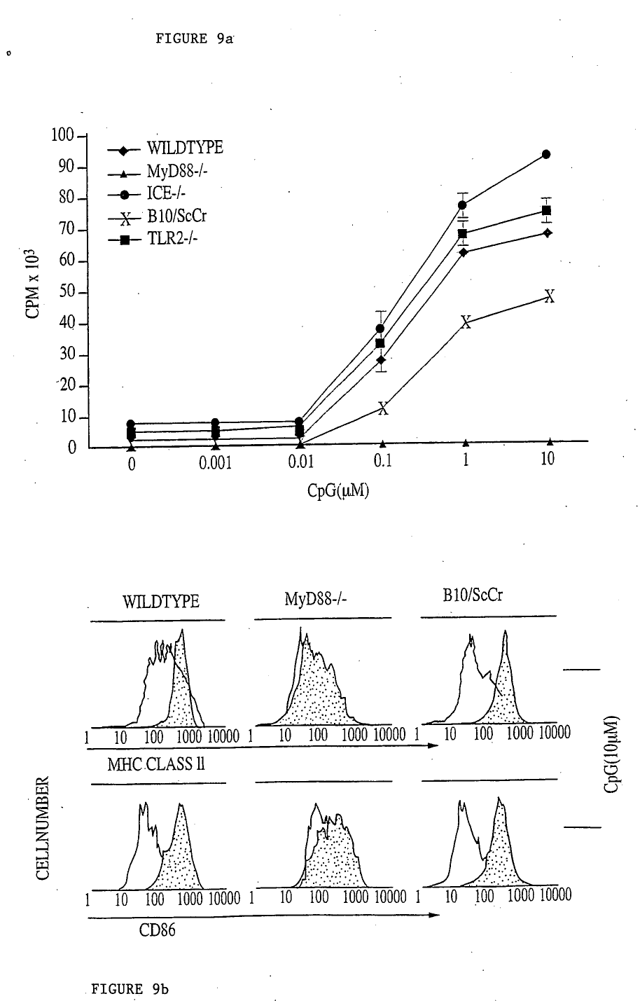

Results shown in FIG. 9A are representative of three independent experiments. B-lymphocytes derived from caspase-1 knock-out mice proliferated in response to CpG comparably to wild type cells (FIG. 9A), suggesting that the effect of the MyD88 deletion is not due to a defect in IL-1/IL-18 mediated signaling. This result indicates that CpG-DNA signals through the receptors of the Toll family. B-cells from two available TLR-deficient mouse strains, the C57BL/10 ScCr strain that carries a spontaneous deletion of the TLR4 gene (Poltorak et al. (1998) Science 282:2085-2088; Qureshi et al. J. Exp. Med. 1999, 189:615-625) and TLR2 knock-out mouse (Takeuchi et al. (1999) Immunity 11:443-451), both proliferated in response to CpG similar to the wild-type cells (FIG. 9A). This result, together with the normal responses of the caspase-1 deficient cells, suggested that a member(s) of the Toll family other than TLR2 or TLR4 is involved in recognition of CPG-DNA.

### Example 11

### CpG and B-cell Expression of CD86 and MHC Class II

The CpG-induced expression of CD86 and upregulation of MHC class-II molecules on B-cells was tested to determine whether these processes are mediated by the MyD88 signaling pathway. B-lymphocytes from MyD88 knock-out mice and wild-type littermate control mice, as well as those from TLR4-deficient mice, were stimulated by CpG-DNA. CD86 and MHC class-II cell surface expression were analyzed by FACS.

B-cells were prepared as above and cultured at 3×10⁶ cells/ml with or without 10 mM CpG for 12 h. After the stimulation, the surface expression of CD86 and MHC class II were analyzed by flow cytometry. Results, shown in FIG. 9B, represent gated B-cells. The shaded area represents stimulated cells, whereas the unshaded area represents untreated controls. As shown in FIG. 9B, CpG-DNA strongly induced expression of CD86 and MHC class-II on B-cells from wild-type and TLR4-deficient mice. By contrast, this induction was completely abrogated in MyD88 deficient B-lymphocytes.

### Example 12

### Cloning of Salmonella Tymphimurium Flagellin and E. coli Flagellin

Full-length Salmonella typhimurium Flagellin and E coli Flagellin were cloned from the respective genomic DNAs and expressed as recombinant proteins in E coli.

Flagellin was expressed alone, or as a fusion protein with antigenic epitopes from ovalbumin (SIINFEKL), tyrosinase-2 protein (TRP2) cloned from murine B16 cells, or the C-terminal fragment of I-Eα protein, which contains the Eα epitope. In addition, all of the recombinant proteins contained a C-terminal 6×-histidine repeat to aid in purification.

Induced bacteria were lysed in a gentle lysis buffer containing Triton-X 100, glycerol, imidazole, NaCl, and Tris, pH=8.0 to maintain the native conformation of the proteins. Fusion proteins were purified by passing filtered lysates over a Nickel-NTA agarose column followed by extensive washes in several buffers containing imidazole. Purified proteins were eluted in 250 mM imidazole, passed twice over a Polymyxin B column to remove contaminating lipopolysaccharide and then dialyzed extensively overnight in PBS at 4° C. The resulting purified proteins were very stable and retain activity at 4° C. for at least a month.

### Example 13

### Flagellin In Vitro Assays

In vitro assays were performed using purified Flagellin fusion proteins as follows:

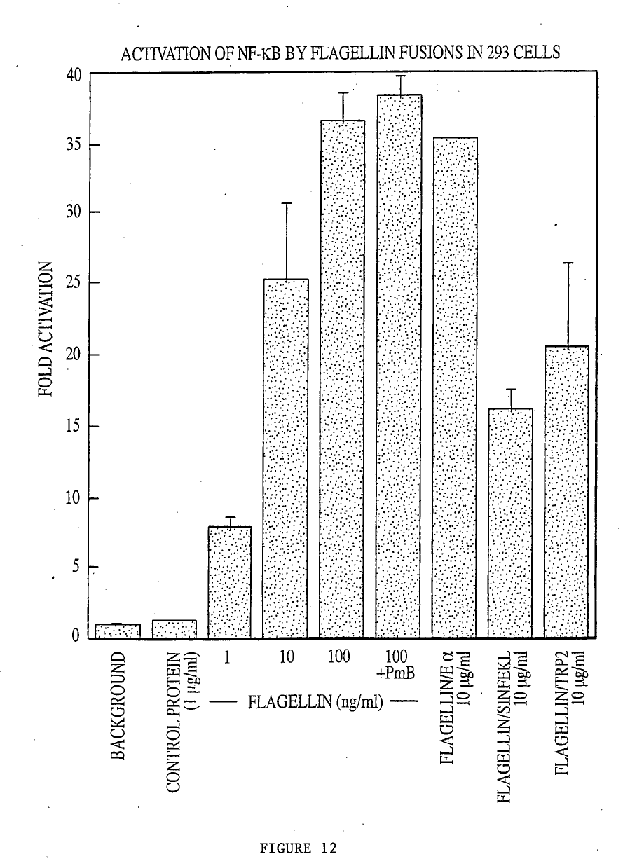

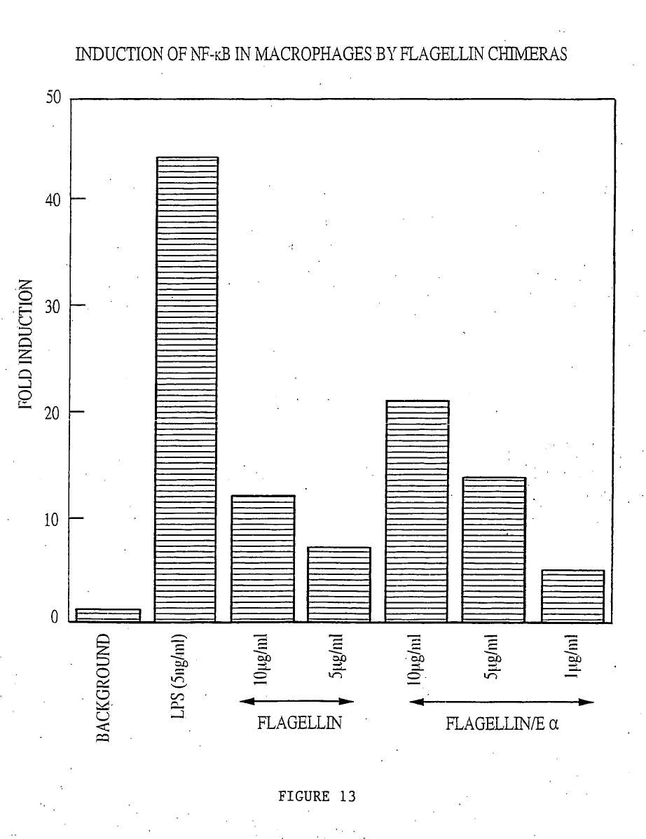

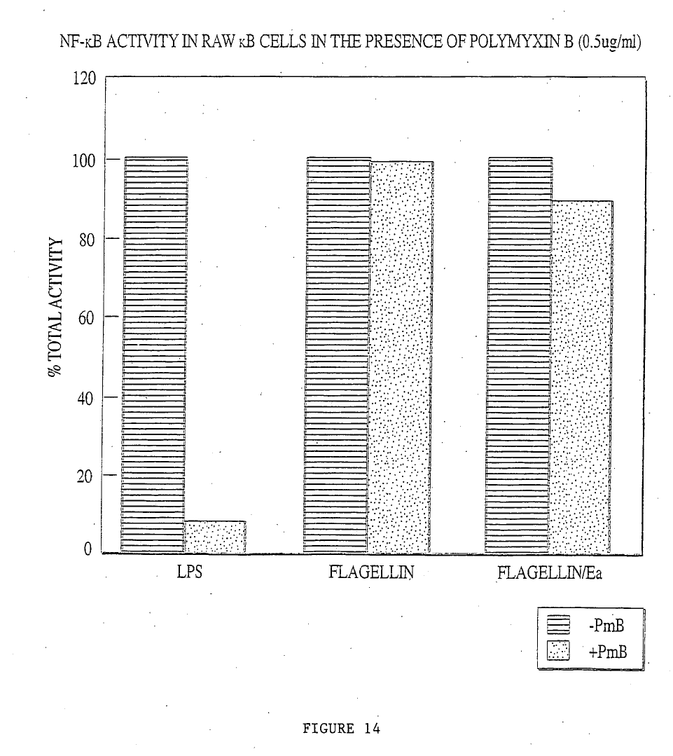

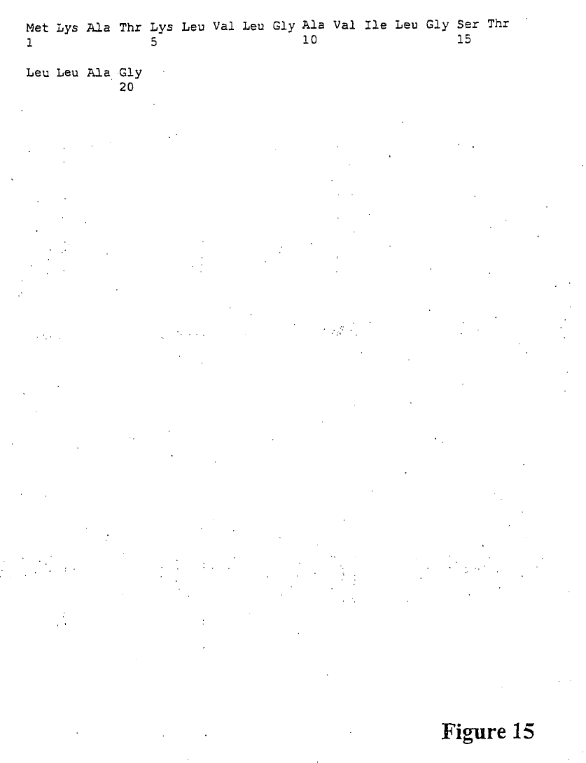

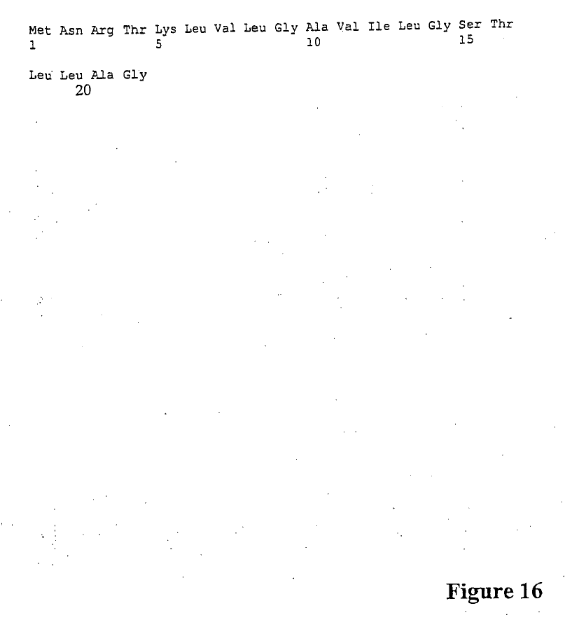

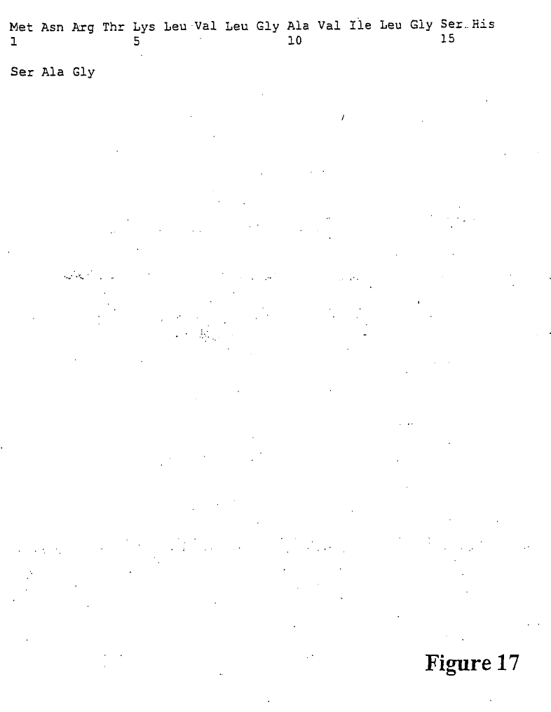

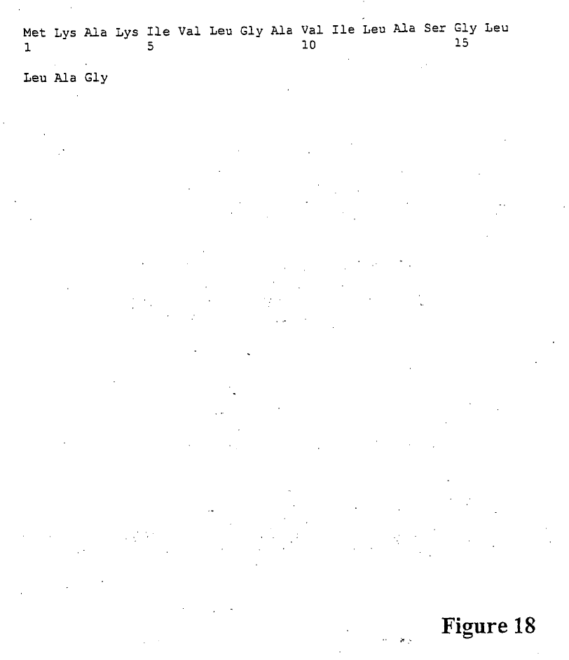

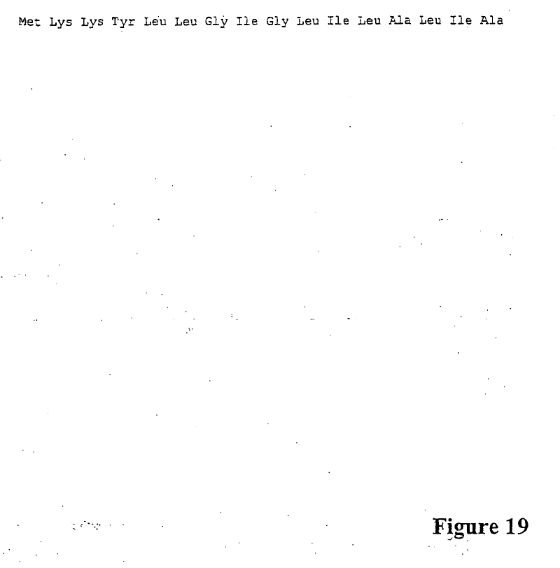

The human 293 cell line and the murine RAW cell line were stably transfected with a reporter gene containing two copies of the IgK NF-κB site driving transcription of luciferase (this construct is referred to as “pBIIxluc”). The resulting cell lines (293LUC and RAWkb) were plated in 24-well dishes and treated 24 hours later with Flagellin fusion proteins or a control protein (lacZ) that was made in the same vector and purified exactly the same way as the Flagellin proteins. Cell lysates were made after 5 hours of treatment and were tested for luciferase activity to indicate induction of NF-κB. The Flagellin proteins significantly induced NF-κB in this assay, particularly in 293 cells whereas the control protein had no effect, as shown in FIGS. 12 and 13. It is believed that this induction was not due to contamination by LPS since polymyxin B did not inhibit the activation in RAWκB cells, and 293LUC cells do not respond to LPS but do respond to Flagellin, as indicated by FIGS. 12 and 14.

The results of the In vitro assays demonstrate that Flagellin fusion proteins retain their ability to stimulate Toll-Like Receptors and can therefore be used for the generation of recombinant Flagellin-Antigen fusion proteins for the purpose of vaccination. In Flagellin-Antigen fusion proteins, Flagellin is believed to stimulate the innate immune system by triggering Toll-Like Receptors, whereas the antigen fused to Flagellin provides epitopes for recognition by T and B lymphocytes.

### Example 14

### CpG and IL-6 Production in Macrophages

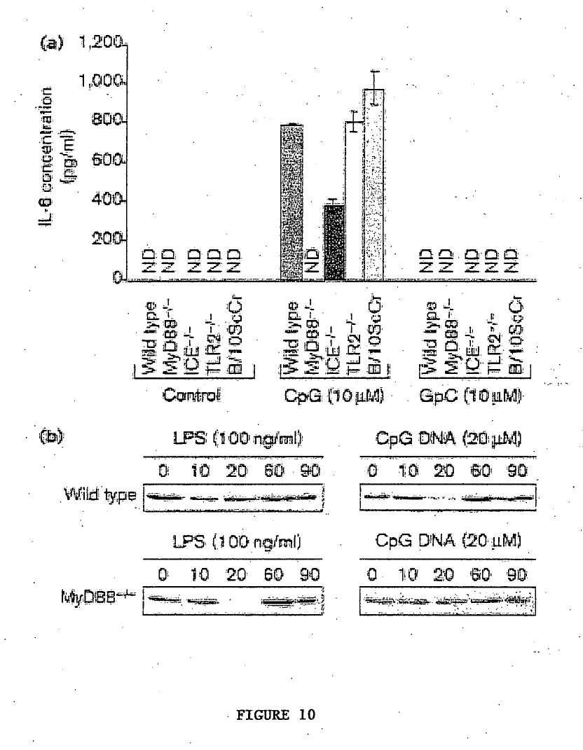

Adherent thioglycollate-elicited peritoneal exudate cells (PECs) from the indicated mouse strains were treated with different stimuli for 24 h. The release of IL-6 into the supernatant was analyzed by specific enzyme-linked immunosorbent assay (ELISA) using anti-mouse IL-6 monoclonal antibodies. As CpG-DNA is also known to have a pronounced stimulatory effect on macrophages (Stacey et al. (2000) Curr. Top. Microbiol. Immunol. 247: 41-58; Lipford et al. (1998) Trends Microbiol. 6: 496-500; Stacey et al. (1996) J. Immunol. 157: 2116-2122), CpG-induced expression of IL-6 by wild-type and MyD88 was examined in deficient macrophages. Cells derived from caspase-1 knock-out mice were used as a control for IL-1-mediated induction of IL-6. The production of IL-6 in response to CpG stimulation was completely abolished in MyD88−/− macrophages, but was normal in caspase-1, TLR2- and TLR4-deficient cells (FIG. 10A). Oligonucleotides consisting of inverted CpG sequence (GpC) were used as a control, and as expected did not induce detectable amounts of IL-6 (FIG. 10A).

### Example 15

### CpG-DNA-Induced IκBA Degradation

We next tested whether activation of the NF-κB signaling pathway is deficient in MyD88−/− macrophages. Peritoneal macrophages were stimulated with CpG-DNA, or LPS as a control, for 0, 10, 20, 60, and 90 minutes and lysed thereafter. For each timepoint, 30 mg total protein was processed for SDS-PAGE and analyzed by immunoblotting for IκBX protein. (FIG. 10B). In wild-type cells, both LPS and CpG-DNA induced NF-κB activation, as evidenced by the degradation of IκB protein (FIG. 10B). In MyD88−/− macrophages, LPS still induced IκB degradation, albeit with delayed kinetics, as is consistent with published observations (Kawai et al. (1999) Immunity 11: 115-122). However, unlike LPS, CpG-DNA did not induce IκB degradation in MyD88−/− macrophages (FIG. 10B). Therefore, while both LPS and CpG-DNA signal through MyD88, the signaling pathways initiated by these stimuli are not identical, reflecting a possibility that different TLRs can activate overlapping but distinct signaling pathways.

### Example 16

### CpG and IL-2 Production in Dendritic Cells

CpG-DNA has been shown to be a potent inducer of DC activation (Sparwasser et al. (1998) Eur. J. Immunol. 28: 2045-2054). DC play a pivotal role in the initiation of the adaptive immune responses (Banchereau et al. (1998) Nature 392: 245-252). Upon interaction with microbe-derived products (PAMPs) in peripheral tissues, DC undergo developmental changes collectively referred to as maturation (Banchereau et al. (1998) Nature 392: 245-252). The hallmark of DC maturation is the induction of cell surface expression of CD80 and CD86 molecules, as well as migration into lymphoid tissues and production of cytokines such as IL-12 (Banchereau et al. (1998) Nature 392: 245-252). We tested therefore, whether the induction of DC maturation by CpG-DNA is mediated by the MyD88 signaling pathway. MyD88−/− animals produce IL-12 when stimulated with CpG oligonucleotides. Wild-type, B10/ScCr, and MyD88−/− bone marrow DC, were prepared from bone marrow suspensions cultured for 5 days in DC Growth Medium (RPMI 5% FC+1% GM-CSF) and stimulated with 10 mm CpG or 10 mm GpC oligonucleotides or left untreated. Supernatants were taken 24 h and 48 h after stimulation and analyzed for IL-12 by ELISA using specific capture and detection antibodies.

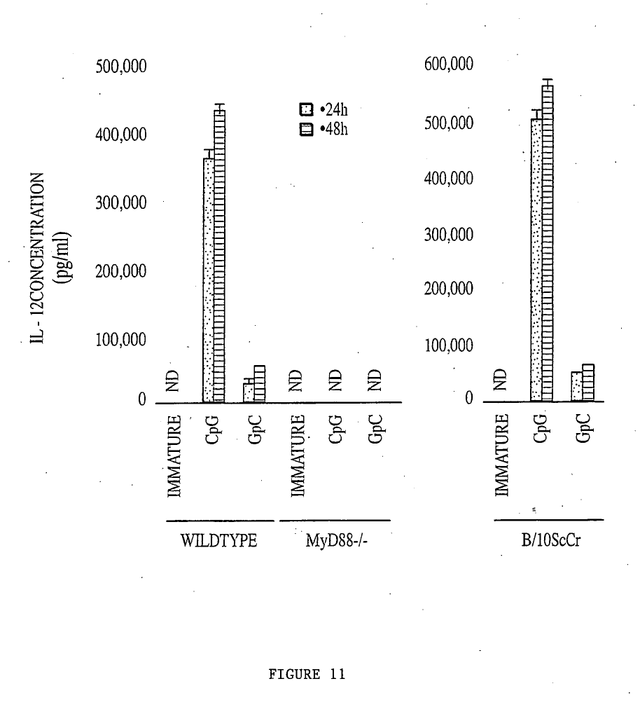

The results, shown in FIG. 11, are from one of three independently performed experiments. Consistent with published reports, CpG-DNA induced secretion of large amounts of IL-12 by DC from the wild-type mice. However, no detectable IL-12 was produced in response to CpG stimulation by DC derived from MyD88 knock-out mice (FIG. 11). As expected, DC from TLR4-deficient mice produced wild-type levels of IL-12 in response to CpG-DNA (FIG. 11).

### Example 17

### CpG/EΔ Chimeric Construct

A non-protein PAMP, CpG, was conjugated to the characterized mouse antigen, Eα, through a PEG polymer linker and/or copolymers of D-lysine and D-glutamate, according to the methods described in U.S. Pat. No. 6,06,0056. A CpG-DNA derivative, comprising CpG₄₀ was used as the non-protein PAMP.

All articles, patents and other materials referred to below are specifically incorporated herein by reference.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

### Example 18

### Cloning of E. Coli FimC

Full-length E. coli FimC was cloned from the genomic DNA and expressed as a recombinant protein in E. coli. The recombinant FimC protein contained a C-terminal 6×-histidine repeat to aid in purification.

FimC was expressed in E. coli and induced with IPTG. The protein was purified by lysis and sonication in buffer containing PBS, 0.5% Triton X-100, 5 mM imidazole, 5% glycerol and 300 mM NaCl. The lysate was passed over a 2 ml Nickel-agarose column followed by washes with 10 ml of the same buffer followed by washes with 5 ml of lysis buffer with 15 mM imidazole and 5 ml of phosphate buffer with 0.1% Triton X-100, 10 mM imidazole. Purified proteins were eluted in PBS, 0.1% Triton X-100 and 400 mM imidazole. The positive fractions were determined by immunoblotting and pooled. Pooled fractions were passed twice over a polymyxin B column and then dialyzed overnight in PBS with 0.1% Triton X-100 to remove imidazole.

### Example 19

### FimC In Vitro Assays

In vitro assays were performed using purified FimC proteins as follows:

The murine RAW macrophage and NIH 3T3 cells lines were stably transfected with a reporter gene containing an NF-κB-dependent firefly luciferase gene. Stimulation of these cells with activators of NF-κB leads to production of luciferase which is measured in cell lysates by use of a luminometer.

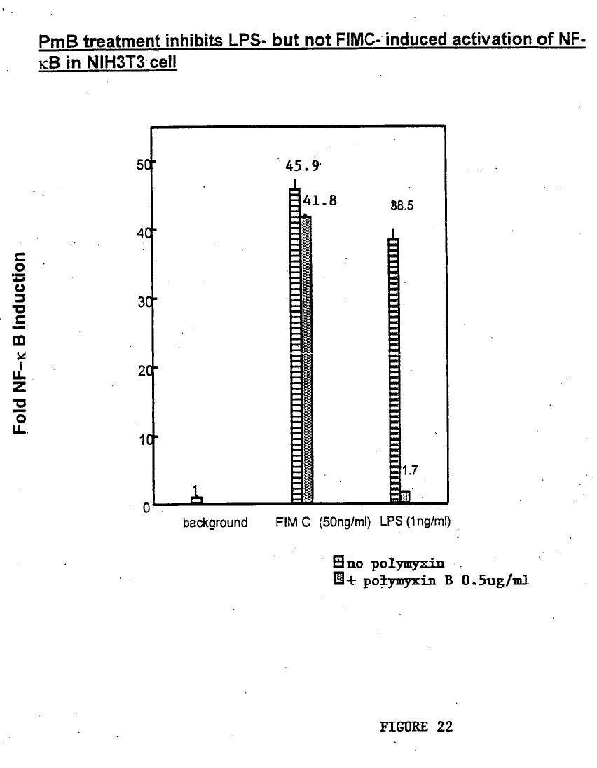

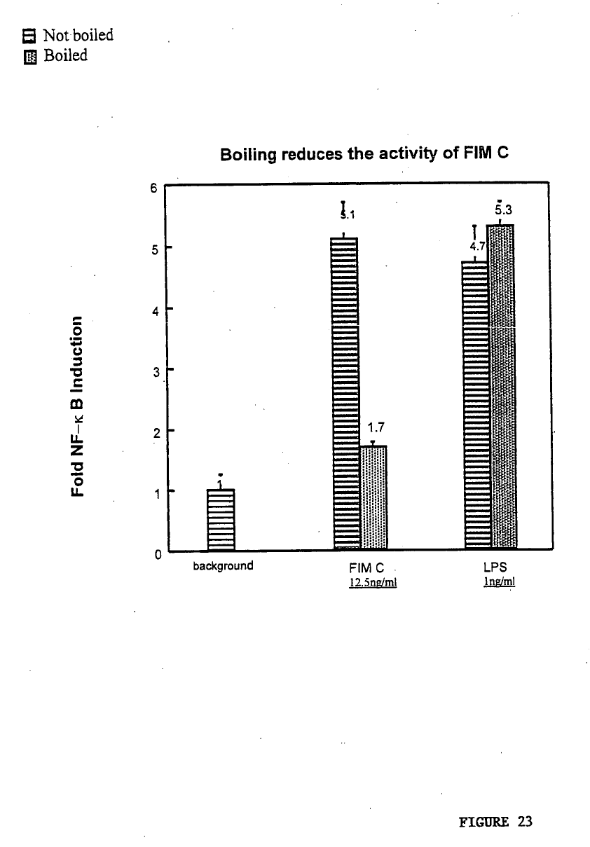

Cells were stimulated with the indicated amounts of FimC, left 5 hours and harvested for luciferase measurement. As a control, RAW macrophage and NIH 3T3 cells were stimulated with LPS in the presence and absence of polymyxin B (PmB). PmB inactivates endotoxin and, as expected, the activation of NF-κB activity in the LPS+PmB sample is diminished by a statistically significant degree. Treatment with PmB, however, does not prevent FimC from activating NF-κB activity to a statistically significant degree (FIG. 22). These results suggest that the activation of NF-κB seen with FimC is not due to contamination of the preparation with endotoxin. Furthermore, boiling FimC eliminates FimC activity (FIG. 23), indicating that the activity depends on an intact conformation of FimC and is not due to LPS contamination.

1-40. (canceled)

41. A fusion protein comprising a pathogen-associated molecular pattern and at least one antigen selected from the group consisting of an avian influenza, a Dengue virus, a Hepatitis C Virus and a Human Papilloma Virus, wherein the pathogen-associated molecular pattern is lipidated and activates a Toll-like receptor 2.

42. The fusion protein of claim 41, wherein the antigen is fused to an amino-terminus of the pathogen-associated molecular pattern.

43. The fusion protein of claim 41, wherein the antigen is fused to a carboxyl-terminus of the pathogen-associated molecular pattern.

44. The fusion protein of claim 41, wherein the lipidated pathogen-associated molecular pattern includes SEQ ID NO: 2.

45. The fusion protein of claim 44, wherein amino acid 21 of SEQ ID NO: 2 is lipidated.

46. The fusion protein of claim 45, wherein a lysine residue at amino acid 78 of SEQ ID NO: 2 is modified.

47. The fusion protein of claim 46, wherein the lysine residue is modified to an amino acid and the amino acid is selected from the group consisting of cysteine, histidine, aspartic acid, tyrosine, tryptophan, and glutamic acid.

48. The fusion protein of claim 46, wherein the lysine residue is modified to a non-naturally occurring amino acid.

49. The fusion protein of claim 41, wherein the antigen is the avian influenza.

50. The fusion protein of claim 41, wherein the antigen is the Dengue virus.

51. The fusion protein of claim 41, wherein the antigen is the Hepatitis C virus.

52. The fusion protein of claim 41, wherein the antigen is the Human Papilloma Virus.

53. The fusion protein of claim 41, further including a linker between the pathogen-associated molecular pattern and the antigen.

54. The fusion protein of claim 53, wherein the linker is a peptide linker.

55. The fusion protein of claim 41, wherein the lipidated pathogen-associated molecular pattern includes SEQ ID NO: 1.

56. The fusion protein of claim 55, further including a leader peptide.

57. The fusion protein of claim 56, wherein the leader peptide is selected from the group consisting of SEQ ID NO: 3, SEQ ID NO: 4, SEQ ID NO: 5, SEQ ID NO: 6 and SEQ ID NO: 7.

58. The fusion protein of claim 41, wherein the lipidated pathogen-associated molecular pattern includes SEQ ID NO: 1 and a leader peptide, wherein the leader peptide is selected from the group consisting of SEQ ID NO: 3, SEQ ID NO: 4, SEQ ID NO: 5, SEQ ID NO: 6 and SEQ ID NO: 7.


SEQUENCE LISTING (15 sequences)

SEQ ID NO: 1 - protein, 4 aa, Artificial Sequence
  lipidation site
  VARIANT (2)...(3): x=any amino acid, preferably serine
  VARIANT 2, 3: Xaa = Any Amino Acid
CXXN

SEQ ID NO: 2 - protein, 78 aa, Escherichia coli
  BLP
MKATKLVLGAVILGSTLLAGCSSNAKIDQLSSDVQTLNAKVDQLSNDVNAMRSDVQAAKDDAARANQRLDNMATKYRK

SEQ ID NO: 3 - protein, 20 aa, Escherichia coli
  BLP leader sequence
MKATKLVLGAVILGSTLLAG

SEQ ID NO: 4 - protein, 20 aa, Erwinia amylovora
  BLP leader sequence
MNRTKLVLGAVILGSTLLAG

SEQ ID NO: 5 - protein, 19 aa, Serratia marcescens
  BLP leader sequence
MNRTKLVLGAVILGSHSAG

SEQ ID NO: 6 - protein, 19 aa, Proteus mirabilis
  BLP leader sequence
MKAKIVLGAVILASGLLAG

SEQ ID NO: 7 - protein, 16 aa, Borrelia burgdorferi
  Outer surface protein A
MKKYLLGIGLILALIA

SEQ ID NO: 8 - DNA, 20 bp, Artificial Sequence
  CpG-DNA
tccatgacgttcctgacgtt

SEQ ID NO: 9 - protein, 73 aa, Ambrosia trifida
  Ra5G ragweed pollen allergen
MKNIFMLTLFILIITSTIKAIGSTNEVDEIKQEDDGLCYEGTNCGKVGKYCCSPIGKYCVCYDSKAICNKNCT

SEQ ID NO: 10 - DNA, 2182 bp, Mus musculus
  misc_feature (405)...(1958): Tyrosinase-Related Kinase Protein 2 (TRP-2)
  misc_feature (945)...(968): T-cell epitope
  misc_recomb (840)...(1040): Region linked to BLP to form fusion protein
gcagcataataagcagtatggctggagcactctgtaaattaactcaattagacagagcctgatttaacaaggaagactggcgagaagctcccctcattaaacctgatgttagaggagcttcggatgaaattaaatcagtgttagttgtttgagtcacataaaattgcatgagcgtgtacacatgtgcacacgtgtaggctctgtgatttaggtgggaattttgagaggagaggaaagggctagaactaaacccaaagaaaaggaaagaagagaagaggaaaggaaagaaaaaagaaaaggcaatttgagtgagtaaaggttccagaactcaggagtggaagacaaggagtaaagtcagacagaaaccaggtgggacgccggccaggcctcccaattaagaaggcatgggccttgtgggatgggggcttctgctgggttgtctgggctgcggaattctgctcagagctcgggctcagtttccccgagtctgcatgaccttggatggcgtgctgaacaaggaatgctgcccgcctctgggtcccgaggcaaccaacatctgtggatttctagagggcagggggcagtgcgcagaggtgcaaacagacaccagaccctggagtggcccttatatccttcgaaaccaggatgaccgtgagcaatggccgagaaaattcttcaaccggacatgcaaatgcacaggaaactttgctggttataattgtggaggctgcaagttcggctggaccggccccgactgtaatcggaagaagccggccatcctaagacggaatatccattccctgactgcccaggagagggagcagttcttgggcgccttagacctggccaagaagagtatccatccagactacgtgatcaccacgcaacactggctggggctgctcggacccaacgggacccagccccagatcgccaactgcagcgtgtatgacttttttgtgtggctccattattattctgttcgagacacattattaggtccaggacgcccctataaggccattgatttctctcaccaagggcctgcctttgtcacgtggcacaggtaccatctgttgtggctggaaagagaactccagagactcactggcaatgagtcctttgcgttgccctactggaactttgcaaccgggaagaacgagtgtgacgtgtgcacagacgactggcttggagcagcaagacaagatgacccaacgctgattagtcggaactcgagattctctacctgggagattgtgtgcgacagcttggatgactacaaccgccgggtcacactgtgtaatggaacctatgaaggtttgctgagaagaaacaaagtaggcagaaataatgagaaactgccaaccttaaaaaatgtgcaagattgcctgtctctccagaagtttgacagccctcccttcttccagaactctaccttcagcttcaggaatgcactggaagggtttgataaagcagacggaacactggactctcaagtcatgaaccttcataacttggctcactccttcctgaatgggaccaatgccttgccacactcagcagccaacgaccctgtgtttgtggtcctccactcttttacagacgccatctttgatgagtggctgaagagaaacaacccttccacagatgcctggcctcaggaactggcacccattggtcacaaccgaatgtataacatggtccccttcttcccaccggtgactaatgaggagctcttcctaaccgcagagcaacttggctacaattacgccgttgatctgtcagaggaagaagctccagtttggtccacaactctctcagtggtcattggaatcctgggagctttcgtcttgctcttggggttgctggcttttcttcaatacagaaggcttcgcaaaggctatgcgcccttaatggagacaggtctcagcagcaagagatacacggaggaagcctagcatgctcctacctggcctgacctgggtagtaactaattacaccgtcgctcatcttgagacaggtggaactcttcagcgtgtgctctttagtagtgatgatgatgatgccttagcaatgacaattatctctagttgctgctttgcttattgtacacagacaaaatgcttgggtcattcaccacggtcaaagtaaggtgtggctagtatatgtgacctttgattag

SEQ ID NO: 11 - protein, 517 aa, Mus musculus
  (181) ... (188) T-cell epitope
  (146) ... (212) Region linked to BLP to form fusion protein
MGLVGWGLLLGCLGCGILLRARAQFPRVCMTLDGVLNKECCPPLGPEATNICGFLEGRGQCAEVQTDTRPWSGPYILRNQDDREQWPRKFFNRTCKCTGNFAGYNCGGCKFGWTGPDCNRKKPAILRRNIHSLTAQEREQFLGALDLAKKSIHPDYVITTQHWLGLLGPNGTQPQIANCSVYDFFVWLHYYSVRDTLLGPGRPYKAIDFSHQGPAFVTWHRYHLLWLERELQRLTGNESFALPYWNFATGKNECDVCTDDWLGAARQDDPTLISRNSRFSTWEIVCDSLDDYNRRVTLCNGTTEGLLRRNKVGRNNEKLPTLKNVQDCLSLQKFDSPPFFQNSTFSFRNALEGFDKADGTLDSQVMNLHNLAHSFLNGTNALPHSAANDPVFVVLHSFTDAIFDEWLKRNNPSTDAWPQELAPIGHNRMYNMVPFFPPVTNEELFLTAEQLGYNYAVDLSEEEAPVWSTTLSVVIGILGAFVLLLGLLAFLQYRRLRKGYAPLMETGLSSKRYTEEA

SEQ ID NO: 12 - protein, 68 aa, Mus musculus
  SITE (37)...(44): T-cell epitope
  TRP-2
LDLAKKSIHPDYVITTQHWLGLLGPNGTQPQIANCSVYDFFVWLHYYSVRDTLLGPGRPYKAIDFSHQ

SEQ ID NO: 13 - protein, 8 aa, Mus musculus
  SITE (1)...(8): T-cell epitope
VYDFFVWL

SEQ ID NO: 14 - protein, 241 aa, Escherichia coli
MSNKNVNVRKSQEITFCLLAGILMFMAMMVAGRAEAGVALGATRVIYPAGQKQEQLAVTNNDENSTYLIQSWVENADGVKDGRFIVTPPLFAMKGKKENTLRILDATNNQLPQDRESLFWMNVKAIPSMDKSKLTENTLQLAIISRIKLYYRPAKLALPPDQAAEKLRFRRSANSLTLINPTPYYLTVTELNAGTRVLENALVPPMGESTVKLPSDAGSNITYRTINDYGALTPKMTGVME

SEQ ID NO: 15 - DNA, 821 bp, Escherichia coli
ccaggttctctttaacctatcagtaattgttcagcagataatgtgataacaggaacaggacagtgagtaataaaaacgtcaatgtaaggaaatcgcaggaaataacattctgcttgctggcaggtatcctgatgttcatggcaatggtggttgccggacgcgctgaagcgggagtggccttaggtgcgactcgcgtaatttatccggcagggcaaaaacaagtgcaacttgccgtgacaaataatgatgaaaatagcacctatttaattcaatcatgggtagaaaatgccgatggtgtaaaggatggccgttttatcgtgacgccacctctgtttgcgatgaagggaaaaaaagagaataccttgcgtattcttgatgcaacaaataaccaattgccacaggatcgggaaagtttattctggatgaatgttaaagcgattccgtcaatggataaatcaaaattgactgagaatacgctacagctcgcaattatcagccgcattaaactgtactatcgtccggctaaattagcgttgccacccgatcaggccgcagaaaaattaagatttcgtcgtagcgcgaattctctgacgctgattaacccgacaccctattacctgacggtaacagagttgaatgccggaactcgggttcttgaaaatgcattggtgcctccaatgggcgaaagcgcggttaaattgccttctgatgcaggaagcaatattacttaccgaacaataaatgattatggcgcacttacccccaaaatgacgggggtaatggaataacgcagggggattttttcgcctaataaaaaaatt